(19) United States
(12) Patent Application Publication
(10) Pub. No.: US 2025/0115607 A1
(43) Pub. Date: Apr. 10, 2025

(54) PYRIDINE DERIVATIVES

(71) Applicant: Hoffmann-La Roche Inc., Little Falls, NJ (US)

(72) Inventors: Guillaume DÉCORET, Eschentzwiller (FR); Katrin GROEBKE ZBINDEN, Liestal (CH); Wolfgang HAAP, Lörrach (DE); Lukas KREIS, Baden (CH); Xavier LUCAS CABRÉ, Basel (CH); Etienne RAUBER, Hundsbach (FR)

(73) Assignee: Hoffmann-La Roche Inc., Little Falls, NJ (US)

(21) Appl. No.: 18/985,716

(22) Filed: Dec. 18, 2024

Related U.S. Application Data
(63) Continuation of application No. PCT/EP2023/067759, filed on Jun. 29, 2023.

(30) Foreign Application Priority Data
Jul. 1, 2022 (EP) 22182508.6

(51) Int. Cl. C07D 471/04; A61K 31/444; A61K 31/501; A61K 31/5383; C07D 519/00

(52) U.S. Cl. CPC C07D 471/04; A61K 31/444; A61K 31/501; A61K 31/5383; C07D 519/00

(57) ABSTRACT

The invention relates to a compound of formula (I)

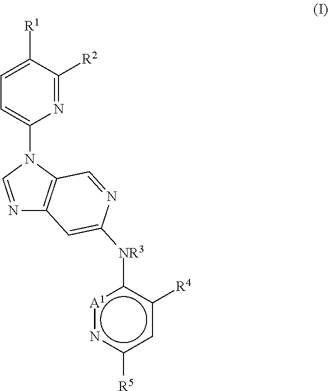

wherein A¹, R¹, R², R³, R⁴ and R⁵ are as defined in the description and in the claims. The compound of formula (I) can be used as a medicament.

### CROSS REFERENCE TO RELATED APPLICATIONS

This application is a continuation of International Application No. PCT/EP2023/067759 filed on Jun. 29, 2023, which claims priority to EP Application No. 22182508.6 filed on Jul. 1, 2022, the disclosures of which are incorporated herein by reference.

### FIELD OF THE INVENTION

The present invention relates to organic compounds useful for therapy and/or prophylaxis in a mammal, and in particular to compounds that modulate SIK activity.

### BACKGROUND OF THE INVENTION

Salt-inducible kinases (SIK) belong to a subfamily of AMP-activated protein kinases (AMPK) called AMPK-related kinases. There are three members, named SIK1, SIK2 and SIK3, that are broadly expressed. Their major biological role is to modify gene expression by controlling the phosphorylation and subcellular localization of two key classes of transcriptional regulatory factors: CRTCs (cAMP-regulated transcriptional coactivators) and class IIa HDACs (Histone deacetylases). Indeed, in basal state, both CRTCs and HDACs are phosphorylated by SIK kinases, and sequestered in the cytoplasm through interactions with their cytoplasmic chaperones 14-3-3. In response to extracellular cues that usually increase intracellular levels of cAMP, the SIK kinases' activity is inhibited, CRTCs and HDACs are no longer phosphorylated and are hence released from 14-3-3. They can therefore translocate into the nucleus and regulate gene expression (reviewed in Wein et al., Trends Endocrinol Metab. 2018 October; 29(10):723-735).

In macrophages, the inhibition of SIK kinases leads to 1) CRTC3 shuttling to the nucleus and increasing the transcription of IL-10; and 2) translocation of HDAC4/5 to the nucleus and subsequent deacetylation of NF-κB resulting in decreased transcription of pro-inflammatory cytokines (Clark et al., Proc Natl Acad Sci USA. 2012 Oct. 16; 109(42):16986-91.).

Macrophages are critical to maintaining tissue homeostasis, mediating inflammation, and promoting the resolution of inflammation. To achieve this diversity of function, macrophages have the ability to “polarize” differently in response to environment cues. The two extreme phenotypes along their activation state continuum are the “M1” or “pro-inflammatory macrophages” and the “M2” or “pro-resolution macrophages”.

Strikingly, the inhibition of intracellular SIK kinases overrides these extracellular macrophage polarization signals and pushes them toward a pro-resolution phenotype. This comes with an increase in IL-10 (by interfering with the SIK-CRTC3 pathway) and a concomitant decrease in TNF-α, IL-12 and IL-6 (by interfering with the SIK-HDAC4/5 and NF-κB pathway). The high levels of IL-10 and low levels of pro-inflammatory cytokines upon SIK inhibition will promote resolution of inflammation. The exploration of the SIK pathway has initially been described in macrophages (Clark et al., Proc Natl Acad Sci USA. 2012 Oct. 16; 109(42):16986-91) and dendritic cells (Sundberg et al., Proc Natl Acad Sci USA. 2014 Aug. 26; 111(34):12468-73) and the therapeutic potential of pan-SIK inhibitors has been confirmed in a mouse LPS (lipopolysaccharide) challenge model (Sundberg et al., ACS Chem Biol. 2016 Aug. 19; 11(8):2105-11) and in colitis models (Fu et al., Inflamm Bowel Dis. 2021 Oct. 20; 27(11):1821-1831). SIKs have since been shown to be important players in the functions of several immune cells, including mast cells (Darling et al., J Biol Chem. 2021 January-June; 296:100428). Importantly, SIK1 is poorly expressed in macrophages and one embodiment of the invention are SIK2/3 inhibitors sparing SIK1, thus limiting potential SIK1-related toxicities.

SIK inhibitors have a high therapeutic potential in diseases that are 1) characterized by pro-inflammatory macrophage influx in the tissues and impaired tissue homeostasis and healing, or 2) where anti-TNF therapies are beneficial (partially or fully) or with insufficient levels of the IL10. Diseases with an inflammatory macrophage signature are e.g. rheumatoid arthritis, juvenile rheumatoid arthritis, NASH, primary sclerosing cholangitis, giant cell vasculitis and inflammatory bowel diseases (“IBD”), atherosclerosis, type 2 diabetes and glomerulonephritis.

Diseases with a proven link to IL-10 and TNF-α are IBD. Genetic alterations that reduce the function of IL-10 (such as SNPs in IL-10 or its receptor) are associated with an increased risk for IBD in humans. In addition, anti-TNF therapies are successful but only a subset of IBD patients are responsive and much of this limited responsiveness is lost over time. The described dual effect of SIK inhibitors (increased IL-10 and decreased TNF-α) make them particularly pertinent for the treatment of IBD.

All three SIK kinase isoforms are expressed broadly in human tissues with the highest expression observed in skin and adipose tissues for SIK1, adipose tissue for SIK2 and testis and brain for SIK3. Similarly to their role in macrophages, SIKs in these cells phosphorylate CRTCs and class II HDACs in response to extracellular signals, which subsequently change the expression of several cellular factors.

In addition to their physiological roles, reports have linked dysregulation of SIK expression to a few diseases. For example, SIK2 has been described as a risk locus for primary sclerosing cholangitis, a fibrotic disease regularly associated with IBD. In addition, SIK2 and SIK3 expression is higher in ovarian and prostate cancers and correlated with poor survival (Miranda et al., Cancer Cell. 2016 Aug. 8; 30(2):273-289; Bon et al., Mol Cancer Res. 2015 April; 13(4):620-635).

As of today many diseases caused by dysregulation of the innate immune system lack efficient therapies and there is a high unmet medical need for new therapies. The present invention relates to a novel compounds that are highly active SIK inhibitors for the treatment of inflammatory, allergic and autoimmune diseases. In addition to inflammation, allergic and autoimmune diseases, SIK inhibitors can thus also be of potential relevance in cancer, metabolic diseases, bone density dysregulation diseases, pigmentation-related diseases or cosmetology, fibrotic diseases and depressive disorders.

### SUMMARY OF THE INVENTION

The invention relates in particular to a compound of formula (I)

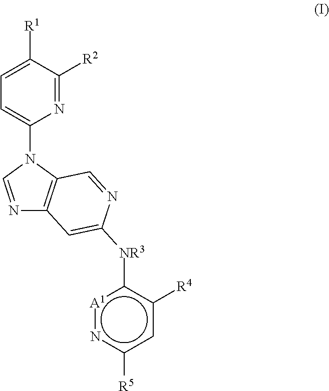

wherein
R¹ is hydroxyalkyl, alkylcarbonyl, cyclopropyl, haloalkyl or cyano;
R² is heteroaryl optionally substituted with one, two or three substituents independently selected from alkyl, haloalkyl, halogen, cyano, haloalkoxy and alkoxy;
R³ is hydrogen or alkyl;
R⁴ is hydrogen or alkyl; or R³ together with R⁴ and the atoms they are attached to, form a five- to seven-membered heterocycloalkyl or heteroaryl ring;
R⁵ is alkyl or haloalkyl; and
A¹ is —CH— or —N—;
or a pharmaceutically acceptable salt thereof.

### DETAILED DESCRIPTION OF THE INVENTION

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups include, but are not limited to, methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, propyl, isopropyl, butyl and isobutyl. Methyl, ethyl, propyl and butyl, like isobutyl, are further particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl include, but are not limited to, cyclopropyl, cyclobutyl, cyclopentyl, cyclohexyl, cycloheptyl and cyclooctyl. Particular examples of “cycloalkyl” are cyclopropyl and cyclobutyl.

The term “heterocycloalkyl”, alone or in combination, denotes a monovalent saturated or partly unsaturated mono- or bicyclic ring system of 4 to 12 ring atoms, comprising 1, 2, or 3 ring heteroatoms selected from N, O and S, the remaining ring atoms being carbon. Bicyclic means consisting of two cycles having one or two ring atoms in common. “Hetercycloylkyl” may comprise a carbonyl group, wherein the carbon is part of the ring system. The ring system can be attached to the remaining compound via an atom selected from C, N, S and O, in particular via a N atom (“N-heterocycloalkyl). Examples of “heterocycloalkyl” include, but are not limited to, morpholino, morpholin-4-yl, pyrrolidinyl, pyrrolidin-1-yl, pyrrolidin-3-yl, piperidinyl, 1-piperidyl, 4-piperidyl, 2-oxopyrrolidin-1-yl, piperazinyl, piperazin-1-yl, azetidinyl, azetidin-1-yl, [3-oxo-piperazin-1-yl], (1,1-dioxo-1,2-thiazolidin-2-yl), (4,5,6,7-tetrahydropyrazolo[4,3-c]pyridin-1-yl), (3-oxo-1,5,6,8-tetrahydrooxazolo[3,4-a]pyrazin-7-yl), [rac-(3aR,6aS)-2,3,3a,5,6,6a-hexahydro-1H-pyrrolo[3,2-b]pyrrol-4-yl], [rac-(3aS,6aR)-2,3,3a,5,6,6a-hexahydro-1H-pyrrolo[3,2-b]pyrrol-4-yl], (4-oxo-6,7-dihydro-5H-pyrazolo[1,5-a]pyrazin-3-yl), (6,7-dihydro-4H-pyrazolo[4,3-c]pyridin-1-yl), (4,7-diazaspiro[2.5]octan-7-yl), (2-oxa-5,8-diazaspiro[3.5]nonan-8-yl), 3-azabicyclo[3.2.0]heptan-3-yl), (5-azaspiro[2.4]heptan-5-yl), (2-azabicyclo[2.2.1]heptan-2-yl), 4-oxa-7-azaspiro[2.5]octan-7-yl, (3-azabicyclo[3.1.0]hexan-3-yl), (6,7-dihydro-4H-pyrazolo[4,3-c]pyridin-1-yl), 2-oxa-7-azaspiro[3.4]octan-7-yl, (2-oxo-1-piperidyl), (2,3-dihydropyridazino[4,5-b][1,4]oxazin-8-yl), pyrrolidin-1-yl, 2-oxo-pyrimidin-4-yl, morpholinoethyl, 2-oxa-5-azaspiro[3.4]octan-5-yl, oxetan-3-yl, (2-oxo-1-piperidyl), 2-oxo-4-piperidyl, 5-oxo-pyrrolidin-3-yl, 2-oxa-5-azaspiro[3.4]octan-5-yl, (7,8-dihydro-5H-pyrano[4,3-c]pyridazin-3-yl), [rac-(4aS,7aR)-4-methyl-2,3,4a,5,7,7a-hexahydropyrrolo[3,4-b][1,4]oxazin-6-yl], 3,4-dihydro-2H-1,4-oxazinyl and [rac-(3aS,6aS)-6-oxo-2,3,3a,4,5,6a-hexahydropyrrolo[2,3-c]pyrrol-1-yl]. A particular example of “heterocycloalkyl” is 3,4-dihydro-2H-1,4-oxazine.

The term “heteroaryl”, alone or in combination, signifies an aromatic mono- or bicyclic ring system with 5 to 12 ring atoms, comprising 1, 2, 3 or 4 heteroatoms each independently selected from N, O and S, the remaining ring atoms being carbon. The ring system can be attached to the remaining compound via an atom selected from C, N, S and O, in particular via a N atom (“N-heteroaryl). Examples of heteroaryl include, but are not limited to, pyrazolyl, pyrazol-1-yl, pyrazol-3-yl, pyrazol-4-yl, pyridinyl, 2-pyridyl, 3-pyridyl, 4-pyridyl, pyridazinyl, pyridazin-3-yl, pyridazin-4-yl, pyrazinyl, pyrazin-2-yl, isoxazolyl, isoxazol-3-yl, isoxazol-4-yl, pyrimidinyl, pyrimidin-5-yl, benzotriazolyl, 1H-benzotriazol-4-yl, furanyl, furyl, 2-furyl, 3-furyl, [6-oxo-1H-pyridazin-5-yl], triazolyl, triazol-1-yl, triazol-2-yl, 2-oxo-4-pyridyl. pyrimidin-2-yl, pyrimidin-5-yl, (1,3,4-oxadiazol-2-yl), (1,3,4-thiadiazol-2-yl), (1,2,4-triazin-3-yl), 2-oxo-pyrimidin-4-yl, (1-methyl-2-oxo-3-pyridyl) and (2,3-dihydropyridazino[4,5-b][1,4]oxazin-8-yl). Particular examples of“heteroaryl” are pyrazol-1-yl, pyrazol-4-yl, pyridazin-3-yl ane pyrimidin-5-yl.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. Particular examples of “alkoxy” are methoxy and ethoxy.

The term “oxy”, alone or in combination, signifies the —O— group.

The term “oxo”, alone or in combination, signifies the ═O group.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens, more particularly two to three halogens. Particular “haloalkyl” are fluoromethyl, fluoroethyl, fluoropropyl, fluorobutyl, difluoromethyl, difluoroethyl, trifluoromethyl and trifluoroethyl.

The term “haloalkoxy”, alone or in combination, denotes an alkoxy group substituted with at least one halogen, particularly substituted with one to five halogens, particularly one to three halogens. Particular “haloalkoxy” are fluoromethoxy, fluoroethoxy and fluoropropyloxy.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “alkylamino” is alkyl group linked to a —NH— group. The term “dialkylamino” denotes two alkyl groups linked to a —N— atom.

The term “sulfonyl”, alone or in combination, signifies the —SO₂— group.

The term “pharmaceutically acceptable salts” denotes salts which are not biologically or otherwise undesirable. Pharmaceutically acceptable salts include both acid and base addition salts. The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid. The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins. Particular pharmaceutically acceptable salts of the compound of formula (I) are for example its formic acid salts.

The term “compound(s) of this invention” and “compound(s) of the present invention” refers to compounds of formula (I) and stereoisomers, tautomers, solvates, and salts (e.g., pharmaceutically acceptable salts) thereof.

Tautomeric forms, i.e. structural isomers which interconvert with the compound of formula (I), in particular in solution, may in some instances exist and are to be understood as being included in the invention.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I).

If desired, racemic mixtures of the compound of the invention may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where an optically pure enantiomer is provided, optically pure enantiomer means that the compound contains >90% of the desired isomer by weight, particularly >95% of the desired isomer by weight, or more particularly >99% of the desired isomer by weight, said weight percent based upon the total weight of the isomer of the compound. A chirally pure or chirally enriched compound may be prepared by chirally selective synthesis or by separation of enantiomers. The separation of enantiomers may be carried out on the final product or alternatively on a suitable intermediate.

Structures depicted herein are also meant to include compounds that differ only in the presence of one or more isotopically enriched atoms. Particular examples of radioisotopes are ²H, ³H, ¹³C, ¹⁴C and ¹⁸F. For example the structures wherein one or more hydrogen atoms are replaced by deuterium or tritium, or one or more carbon atoms are replaced by a ¹³C- or ¹⁴C-enriched carbon are within the scope of this invention.

The invention thus relates to:

A compound according to the invention wherein R¹ is hydroxyalkyl or alkylcarbonyl;

A compound according to the invention wherein R² is hydroxyethyl or methylcarbonyl;

A compound according to the invention wherein R² is cyano, haloalkyl or haloalkoxy;

A compound according to the invention wherein R² is cyano, difluoromethyl or difluoromethoxy;

A compound according to the invention wherein R³ is hydrogen or methyl, and R⁴ is hydrogen; or R³ together with R⁴ and the atoms they are attached to, form a five- to seven-membered heterocycloalkyl or heteroaryl ring;

A compound according to the invention wherein R³ is hydrogen or methyl, and R⁴ is hydrogen; or R³ together with R⁴ and the atoms they are attached to, form 3,4-dihydro-2H-1,4-oxazine;

A compound according to the invention wherein R³ is hydrogen or alkyl, and R⁴ is hydrogen;

A compound according to the invention wherein R³ together with R⁴ and the atoms they are attached to, form a five- to seven-membered heterocycloalkyl or heteroaryl ring;

A compound according to the invention wherein R³ together with R⁴ and the atoms they are attached to, form 3,4-dihydro-2H-1,4-oxazine

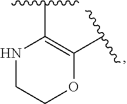 [SMILES: *C1=C(*)OCCN1]

and wherein the double bond of 3,4-dihydro-2H-1,4-oxazine is part of the aromatic system of the pyridazine of the compound of formula (I);

A compound according to the invention wherein R⁵ is methyl or difluoromethyl;

A compound according to the invention wherein R⁵ is alkyl;

A compound according to the invention wherein R⁵ is methyl;

A compound according to the invention wherein A¹ is —N—; and

A compound according to the invention wherein A¹ is —CH—.

The invention further relates to a compound of formula (I) selected from
1-[3-acetyl-6-[6-[(6-methylpyridazin-3-yl)amino]imidazo[4,5-c]pyridin-3-yl]-2-pyridyl]-5-methyl-pyrazole-3-carbonitrile;
1-[3-(1-hydroxyethyl)-6-[6-[(6-methylpyridazin-3-yl)amino]imidazo[4,5-c]pyridin-3-yl]-2-pyridyl]-5-methyl-pyrazole-3-carbonitrile;
1-[3-(1-hydroxyethyl)-6-[6-[(6-methyl-3-pyridyl)amino]imidazo[4,5-c]pyridin-3-yl]-2-pyridyl]-5-methyl-pyrazole-3-carbonitrile;
1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanol;
1-[3-acetyl-6-[6-[methyl-(6-methylpyridazin-3-yl)amino]imidazo[4,5-c]pyridin-3-yl]-2-pyridyl]-5-methyl-pyrazole-3-carbonitrile;
1-[3-acetyl-6-[6-(3-methyl-6,7-dihydropyridazino[4,3-b][1,4]oxazin-8-yl)imidazo[4,5-c]pyridin-3-yl]-2-pyridyl]-5-methyl-pyrazole-3-carbonitrile; and
1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanol;
   or a pharmaceutically acceptable salt thereof.

The invention further relates to a compound of formula (I) selected from
1-[3-acetyl-6-[6-[(6-methylpyridazin-3-yl)amino]imidazo[4,5-c]pyridin-3-yl]-2-pyridyl]-5-methyl-pyrazole-3-carbonitrile;
1-[3-(1-hydroxyethyl)-6-[6-[(6-methylpyridazin-3-yl)amino]imidazo[4,5-c]pyridin-3-yl]-2-pyridyl]-5-methyl-pyrazole-3-carbonitrile; and
1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanol;
   or a pharmaceutically acceptable salt thereof.

The synthesis of the compound of formula (I) can, for example, be accomplished according to the non-exhaustive procedures described below in general schemes 1-6 or according to methods known to those skilled in the art. In some instances, the sequence of the reaction steps can be modified and the individual steps of the different schemes can be combined in different ways as disclosed herein.

### Scheme 1

In scheme 1, the synthesis of a compound of formula (I-b) starting from a compound of formula (I-a) is described. In the scheme below A¹, R², R³, R⁴ and R⁵ are as defined above. The compound of formula (I-a) is a compound of formula (I) as described herein, wherein R¹ is methylcarbonyl. The compound of formula (I-b) is a compound of formula (I) as described herein, wherein R¹ is hydroxyethyl.

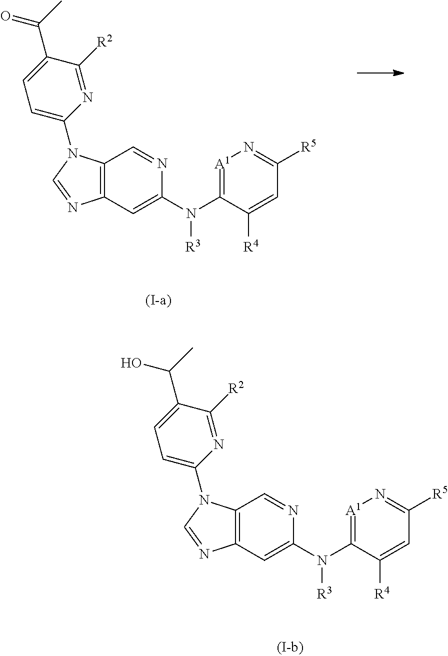

Step A: A compound of formula (I-a) can be converted to a compound of formula (I-b) with a reducing agent such as NaBH₄ in MeOH with a cosolvent such as THF, 1,4-dioxane, EtOH, MeOH, DCM at a temperature between around −40° C. and RT.

### Scheme 2

In the scheme below A¹, R², R³, R⁴ and R⁵ are as defined above. The compound of formula (I-a) is a compound of formula (I) as described herein, wherein R¹ is methylcarbonyl.

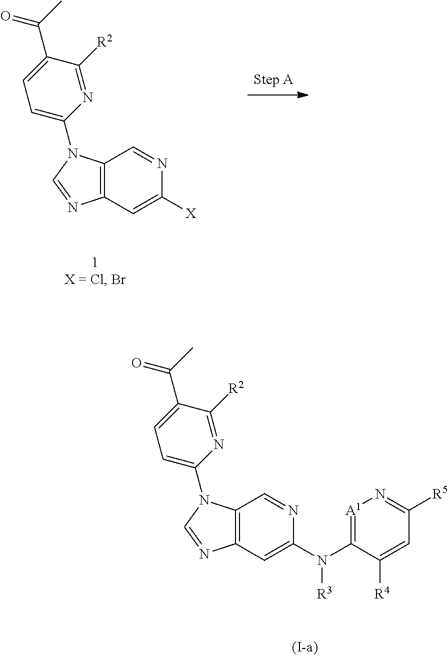

Step A: Intermediate 1 can be converted to a compound of formula (I-a) via a Buchwald-Hartwig coupling of intermediate 3 with an aminopyridazine reactant using a suitable base such as for instance Cs₂CO₃, K₂CO₃ or K₃PO₄ and a suitable palladium catalyst such as for instance t-BuXPhos-Pd-G3 or [tBuBrettPhos Pd(allyl)]OTf in a suitable solvent such as 1,4-dioxane, optionally with water as cosolvent, at temperatures between around 80° C. and around 100° C.

### Scheme 3

In the scheme below R² is as described herein.

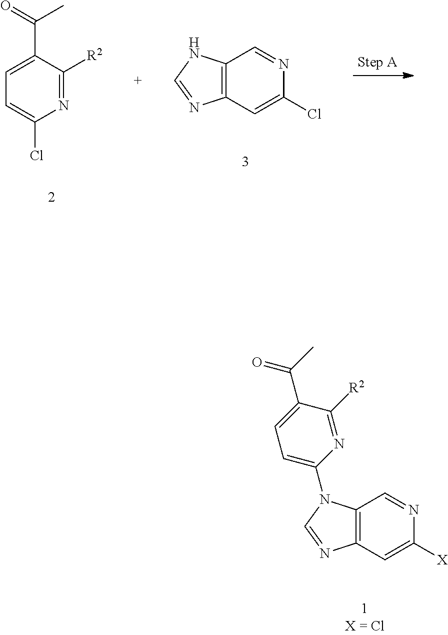

Step A: Intermediate 2 and 3 can be reacted to give an intermediate 1 in the presence of a suitable base such as for instance DBU, K₂CO₃ or Cs₂CO₃ in a suitable solvent such as for instance 1,4-dioxane or DMSO at a temperature of around 80° C.

### Scheme 4

In the scheme below A¹, R², R³, R⁴ and R⁵ are as defined above. The compound of formula (I-a) is a compound of formula (I) as described herein, wherein R¹ is methylcarbonyl.

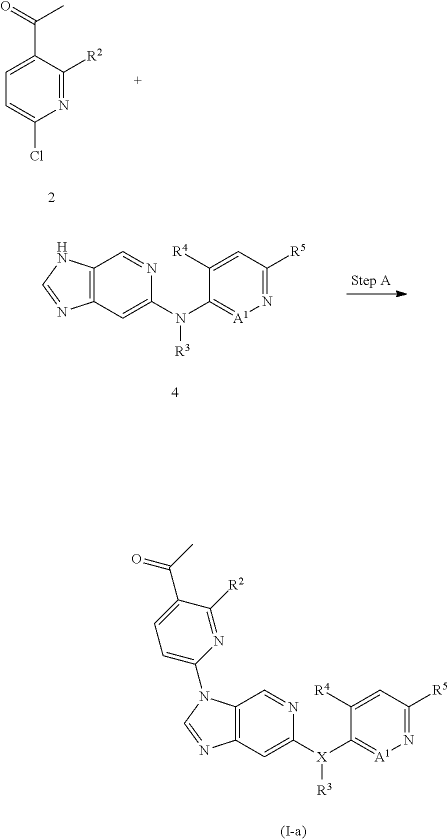

Step A: Intermediate 2 and 4 can be reacted to give the compound of formula (I-a) in the presence of a suitable base such as for example DBU, K₂CO₃ or Cs₂CO₃ in a suitable solvent such as for example 1,4-dioxane, DMF, NMP, DMA or DMSO, optionally with water as cosolvent, at temperatures between around 50° C. and around 80° C.

### Scheme 5

In the scheme below R² is as described herein.

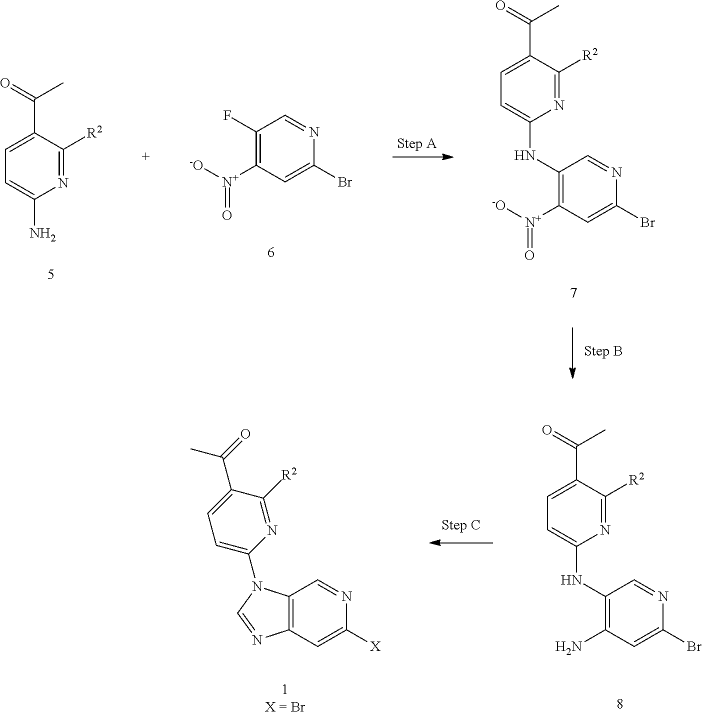

Step A: An intermediate 7 can be obtained by reacting an aniline 5 and a fluoro or chloropyridine 6 in a solvent such as DMF, NMP or DMA in the presence of a deprotonating agent such as for instance NaH at temperatures between around −10° C. and RT.

Step B: An aniline intermediate 8 can be obtained by reduction of the corresponding nitro precursor 7 using a reducing agent such as activated Zn in a solvent such as EtOH or water in the presence of HOAc at a temperature of around 50° C.

Step C: An intermediate 1 can be obtained for aniline intermediate 8 by condensation with trimethoxymethane, optionally in the presence of TsOH, in a solvent such as MeOH at temperatures between around 80° C. and around 120° C.

### Scheme 6

In scheme 6, the synthesis of intermediate 5a is described. The compound of formula 5a is a compound of formula 5, wherein R² is methyl-pyrazolyl optionally substituted with substituent Ra selected from carbonitrile, alkyl or haloalkyl, or substituent R^{b} selected from hydrogen, alkyl and haloalkyl.

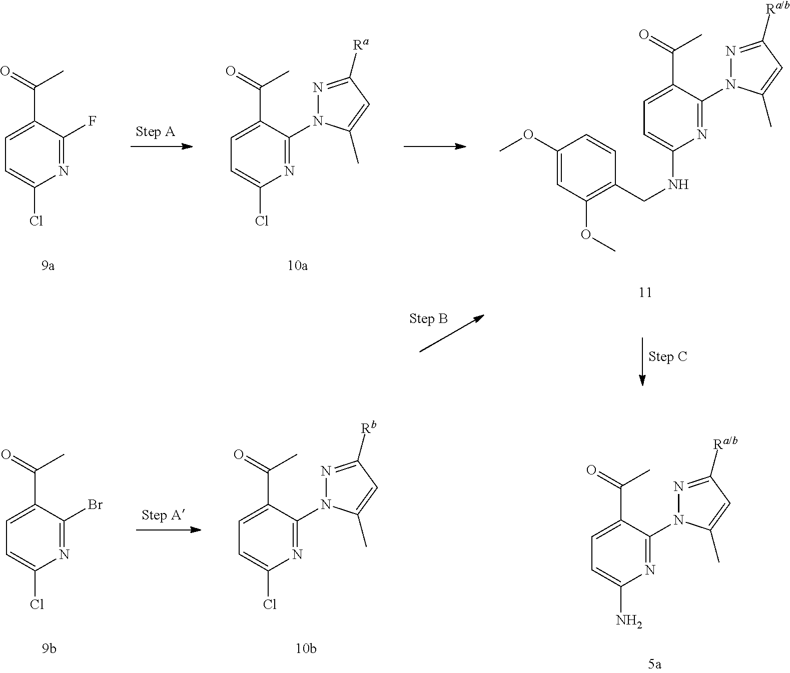

Step A: An intermediate 10a can be obtained by reacting building block 9a with a suitable pyrazole building block in a suitable solvent such as 1,4-dioxane, THF, DMF, DMA, NMP or DMSO in the presence of a suitable base such as DBU at a temperature of around 80° C.

Step A¹: Intermediate 10b can be obtained by reacting starting material 9b with a suitable pyrazole boronic ester in the presence of a suitable base such as K₂CO₃ and a suitable catalyst such as Pd(dppf)Cl₂·CH₂Cl₂ in a solvent such as 1,4-dioxane, optionally with water as cosolvent, at a temperature of around 80° C.

Step B: An intermediate 11 can be obtained by reacting an intermediate 10 with (2,4-dimethoxybenzyl)amine in a suitable solvent such as for example 1,4-dioxane, THF, DMF, DMA, NMP or DMSO in the presence of a suitable base such as for example DIPEA at temperatures between RT and around 80° C.

Step C: An intermediate 5a can be obtained by deprotecting an intermediate 11 with a strong acid such as for instance TFA in a suitable solvent such as for example DCM at RT.

The invention thus also relates to a process for the preparation of a compound according to the invention, comprising one of the following steps:
(a) the reaction of a compound of formula (B1)

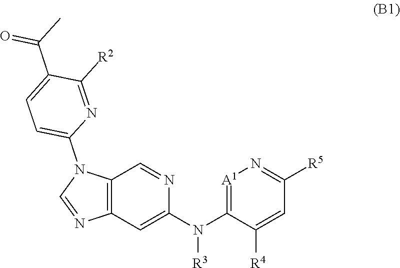

with a suitable reducing agent in presence of a suitable solvent,
(b) the reaction of a compound of formula (B2)

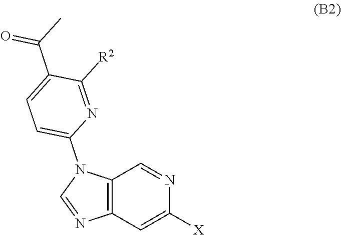

with a compound of formula (B3)

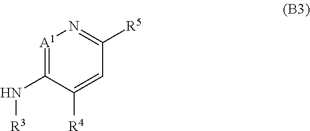

in presence of a suitable base, a suitable catalyst and a suitable solvent, or
(c) the reaction of a compound of formula (B4)

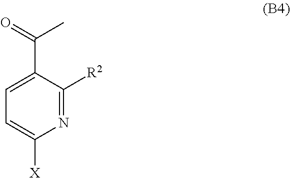

with a compound of formula (B5)

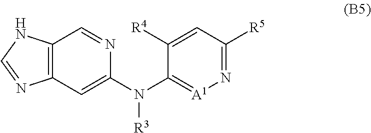

in presence of a suitable base.

In the above process, X is halogen; A¹, R², R³, R⁴ and R⁵ are as described herein.

In the reaction of step (a) the reducing agent can be for example LiBH₄ or NaBH₄.

In the reaction of step (a) the solvent can be for example MeOH optionally with a suitable cosolvent. The cosolvent can be for example THF, dioxane, EtOH, dichloromethane or a mixture thereof.

Convenient conditions for step (a) are between around −60° C. and around room temperature, in particular between around −40° C. and room temperature.

Conveniently, the conditions for step (a) are NaBH₄ as reducing agent, MeOH as solvent with a suitable cosolvent and a reaction temperature of between around −40° C. and room temperature.

In the reaction of step (b) the base can be for example Cs₂CO₃, K₂CO₃ or K₃PO₄.

In the reaction of step (b) the catalyst can be for example a suitable Pd catalyst, in particular t-BuXPhos-Pd-G3 or [tBuBrettPhos Pd(allyl)]OTf.

In the reaction of step (b) the solvent can be for example 1,4-dioxane, THF, DMA, DMF, NMP, toluene, xylene, water or a mixture thereof.

Convenient conditions for step (b) can be between around 60° C. and 120° C., in particular around 70° C. and around 110° C., more particular between around 80° C. and around 100° C.

Conveniently, the conditions for step (b) are Cs₂CO₃ or K₂CO₃ as base, t-BuXPhos-Pd-G3 or [tBuBrettPhos Pd(allyl)]OTf as catalyst and 1,4-dioxane as solvent at a reaction temperature of between around 80° C. and around 100° C.

In the reaction of step (c) the base can be for example DBU, K₂CO₃ or Cs₂CO₃.

In the reaction of step (c) the solvent can be for example 1,4-dioxane, DMF, NMP, DMA or DMSO.

Convenient conditions for step (c) can be between around 30° C. and 120° C., in particular around 40° C. and around 100° C., more particular between around 50° C. and around 80° C.

Conveniently, the conditions for step (c) are Cs₂CO₃ or K₂CO₃ as base, t-BuXPhos-Pd-G3 or [tBuBrettPhos Pd(allyl)]OTf as catalyst and 1,4-dioxane as solvent at a reaction temperature of between around 80° C. and around 100° C.

The invention also relates to a compound according to the invention when manufactured according to a process of the invention.

The invention also relates in particular to:
A compound of formula (I) as describe herein or a pharmaceutically acceptable salt thereof, for use as therapeutically active substance;
A pharmaceutical composition comprising a compound of formula (I) as described herein or a pharmaceutically acceptable salt thereof, and a therapeutically inert carrier; The use of a compound of formula (I) as described herein or a pharmaceutically acceptable salt thereof, for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, in particular inflammatory bowel diseases (IBD);
The use of a compound of formula (I) as described herein or a pharmaceutically acceptable salt thereof, for the preparation of a medicament for the treatment or prophylaxis rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, in particular inflammatory bowel diseases (IBD);
A compound of formula (I) as described herein or a pharmaceutically acceptable salt thereof, for use in the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, in particular inflammatory bowel diseases (IBD); and
A method for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, which method comprises administering an effective amount of a compound of formula (I) as described herein or a pharmaceutically acceptable salt thereof, to a patient in need thereof.

### Pharmaceutical Compositions

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal, epidural and intranasal, and if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention will now be illustrated by the following examples which have no limiting character.

### EXAMPLES

### Abbreviations

[tBuBrettPhos Pd(allyl)]OTf allyl(2-di-tert-butylphosphino-3,6-dimethoxy-2′,4′,6′-triisopropyl-1,1′-biphenyl)palladium(II) triflate (CAS #1798782-15-6)
ATP adenosine triphosphate
aq. aqueous
BINAP (2,2′-bis(diphenylphosphino)-1,1′-binaphthyl)
Boc tert-butyloxycarbonyl
DBU 1,8-diazabicyclo[5.4.0]undec-7-ene
DIPEA N,N-diisopropylethylamine
DMF N,N-dimethylformamide
DMSO dimethyl sulfoxide
dppf 1,1′-ferrocenediyl-bis(diphenylphosphine)
dtbpy 4,4′-Di-tert-butyl-2,2′-dipyridyl
equiv. equivalents
ESI electrospray ionization
Et ethyl
EtOAc ethyl acetate
EtOH ethanol
FA formic acid
HPLC high pressure liquid chromatography
LC-MS high-performance liquid chromatography
Me methyl
MeOH methanol
Ms methanesulfonyl
NMR nuclear magnetic resonance
PE petroleum ether
psi pounds per square inch
RT room temperature
sat. saturated
SFC supercritical fluid chromatography
sol. solution
TBD triazabicyclodecene
TBDMS tert-butyldimethylsilyl
[tBuBrettPhos Pd(allyl)]OTf trifluoromethanesulfonate allyl[(2-di-tert-butylphosphino-3,6-dimethoxy-2′,4′,6′-triisopropyl-1,1′-biphenyl)-2-(2′-amino-1,1′-biphenyl)]palladium(II) (CAS #1798782-15-6)
t-BuXPhos-Pd-G3 [(2-Di-tert-butylphosphino-2′,4′,6′-triisopropyl-1,1′-biphenyl)-2-(2′-amino-1,1′-biphenyl)]palladium(II) methanesulfonate (CAS #1447963-75-8)
TEA triethylamine
Tf triflyl
TFA trifluoroacetic acid
TFAA trifluoroacetic anhydride
THF tetrahydrofuran

### Example 1

### 1-[3-Acetyl-6-[6-[(6-methylpyridazin-3-yl)amino]imidazo[4,5-c]pyridin-3-yl]-2-pyridyl]-5-methyl-pyrazole-3-carbonitrile

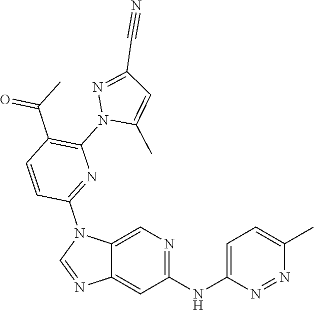 [SMILES: CC(=O)c1ccc(-n2cnc3cc(Nc4ccc(C)nn4)ncc32)nc1-n1nc(C#N)cc1C]

### Step 1: 1-[3-Acetyl-6-(6-chloroimidazo[4,5-c]pyridin-3-yl)-2-pyridyl]-5-methyl-pyrazole-3-carbonitrile

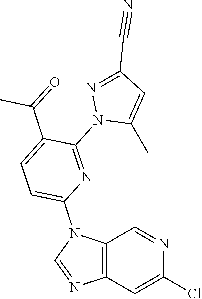 [SMILES: CC(=O)c1ccc(-n2cnc3cc(Cl)ncc32)nc1-n1nc(C#N)cc1C]

Under an argon atmosphere, 6-chloro-3H-imidazo[4,5-c]pyridine (CAS 2589-11-9, 100 mg, 0.65 mmol, 1 equiv.) was dissolved in dimethyl sulfoxide (5 mL). Then, 1-(3-acetyl-6-chloro-2-pyridyl)-5-methyl-pyrazole-3-carbonitrile (ex. 3, step 1; 254 mg, 0.98 mmol, 1.5 equiv.) and 1,8-diazabicyclo[5.4.0]undec-7-ene (CAS 6674-22-2; 109.0 mg, 108 μL, 0.72 mmol, 1.1 equiv.) were added. The reaction mixture was stirred at RT overnight. More 1,8-diazabicyclo[5.4.0]undec-7-ene (109 mg, 108 μL, 0.72 mmol, 1.1 equiv.) was added. The reaction mixture was stirred at 80° C. for 1 hour, then cooled to RT. It was partitioned between water and EtOAc and extracted. The organic layer was dried over MgSO₄, filtered and concentrated in vacuo. The crude product was purified by reversed phase chromatography using water/acetonitrile as eluent to provide a mixture of the two regioisomers (219 mg): The regioisomers were separated by SFC chromatography using 35% MeOH to provide the title compound (39 mg, 16%) as brown solid. LC-MS: m/z=378.1 [M+H]⁺, ESI pos.

### Step 2: 1-[3-Acetyl-6-[6-[(6-methylpyridazin-3-yl)amino]imidazo[4,5-c]pyridin-3-yl]-2-pyridyl]-5-methyl-pyrazole-3-carbonitrile, Formic Acid

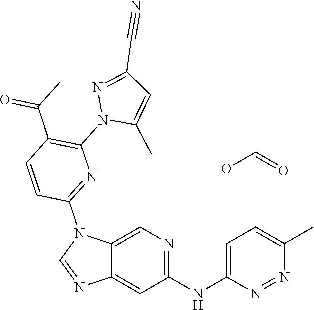 [SMILES: CC(=O)c1ccc(-n2cnc3cc(Nc4ccc(C)nn4)ncc32)nc1-n1nc(C#N)cc1C ; O=CO]

In a sealed tube, 1-[3-acetyl-6-(6-chloroimidazo[4,5-c]pyridin-3-yl)-2-pyridyl]-5-methyl-pyrazole-3-carbonitrile (41 mg, 0.11 mmol, 1 equiv.) was dissolved in 1,4-dioxane (4 mL). (6-methylpyridazin-3-yl)amine (23.7 mg, 0.22 mmol, 2 equiv.), Cs₂CO₃ (70.7 mg, 0.22 mmol, 2 equiv.) and water (29.3 mg, 29.3 uL, 1.63 mmol, 15 equiv.) were added. The reaction mixture was flushed with argon, then t-BuXPhos-Pd-G3 (17.2 mg, 0.022 mmol, 0.2 equiv.) was added. The reaction mixture was stirred at 90° C. After 2.5 hours, t-BuXPhos-Pd-G3 (17.24 mg, 0.022 mmol, 0.20 equiv.) was added again, and stirring was continued for 1 hour at 90° C. The reaction mixture was cooled to RT. One spatula of metal scavenger SiliaMetS® Thiol was added. The reaction mixture was stirred at RT over 30 minutes and filtered, washing the residue on the filter with MeOH/DCM/FA (90/10/0.05). The filtrate was concentrated. The crude product was purified by silica gel chromatography using an DCM/MeOH gradient as eluent and then a reverse phase chromatography using a gradient of 1% aqueous formic acid/acetontrile as eluent to obtain the title compound (4.5 mg, 8%) as light yellow lyophilized powder. LC-MS: m/z=453.3 [M+H]⁺, ESI pos.

### Example 2

### 1-[3-(1-Hydroxyethyl)-6-[6-[(6-methylpyridazin-3-yl)amino]imidazo[4,5-c]pyridin-3-yl]-2-pyridyl]-5-methyl-pyrazole-3-carbonitrile

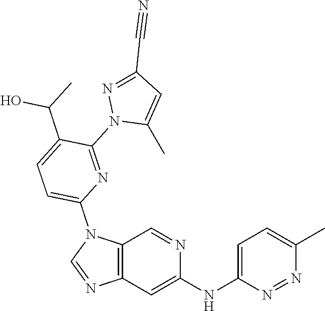 [SMILES: Cc1ccc(Nc2cc3ncn(-c4ccc(C(C)O)c(-n5nc(C#N)cc5C)n4)c3cn2)nn1]

Under an argon atmosphere 1-[3-acetyl-6-[6-[(6-methylpyridazin-3-yl)amino]imidazo[4,5-c]pyridin-3-yl]-2-pyridyl]-5-methyl-pyrazole-3-carbonitrile (example 1; 234 mg, 0.14 mmol, 1 equiv.) was dissolved in methanol (3.5 mL). NaBH₄ (15.9 mg, 0.42 mmol, 3 equiv.) was added and the reaction mixture was stirred at RT over 30 minutes. The reaction mixture was diluted with water. The crude product was purified by reversed phase chromatography using a 0.1% aqueous trimethylamine/acetonitrile gradient as eluent to provide the title compound (15 mg, 23%) as lyophilized powder. LC-MS: m/z=453.3 [M+H]⁺, ESI pos.

### Example 3

### 1-[3-(1-Hydroxyethyl)-6-[6-[(6-methyl-3-pyridyl)amino]imidazo[4,5-c]pyridin-3-yl]-2-pyridyl]-5-methyl-pyrazole-3-carbonitrile

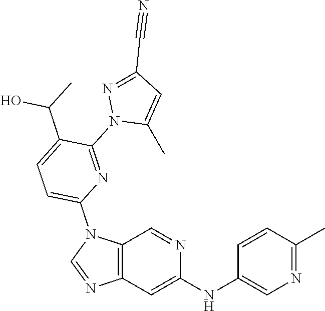 [SMILES: Cc1ccc(Nc2cc3ncn(-c4ccc(C(C)O)c(-n5nc(C#N)cc5C)n4)c3cn2)cn1]

### Step 1: 1-(3-Acetyl-6-chloro-2-pyridyl)-5-methyl-pyrazole-3-carbonitrile

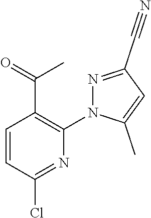 [SMILES: CC(=O)c1ccc(Cl)nc1-n1nc(C#N)cc1C]

A solution of 1-(6-chloro-2-fluoro-3-pyridyl)ethanone (CAS #1260663-13-5; 5 g, 28.81 mmol, 1 equiv.), 5-methyl-1H-pyrazole-3-carbonitrile (2.93 g, 27.4 mmol, 0.95 equiv.) and DIPEA (14.3 mL, 86.42 mmol, 3 equiv.) in DMSO (50 mL) was stirred at 80° C. for 4 hours. The reaction mixture was cooled to RT, poured into H₂O and extracted with EtOAc. The combined organic layers were washed with brine and concentrated. The crude product was purified by flash chromatography using a petroleum ether/EtOAc gradient as eluent to obtain the title compound (5.3 g, 71% yield) as yellow oil. LC-MS: m/z=261.1 [M+H]⁺, ESI pos.

### Step 2: 1-[3-Acetyl-6-[(2,4-dimethoxybenzyl)amino]-2-pyridyl]-5-methyl-pyrazole-3-carbonitrile

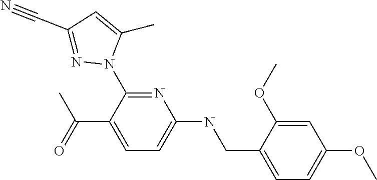 [SMILES: COc1ccc(CNc2ccc(C(C)=O)c(-n3nc(C#N)cc3C)n2)c(OC)c1]

Under an argon atmosphere, 1-(3-acetyl-6-chloro-2-pyridyl)-5-methyl-pyrazole-3-carbonitrile (1.14 g, 4.37 mmol, 1 equiv.) was dissolved in DMSO (8.6 mL). DIPEA (1.9 mL, 10.9 mmol, 2.5 equiv.) and (2,4-dimethoxybenzyl)amine (803 mg, 723 μL, 4.8 mmol, 1.1 equiv.) were added. The reaction mixture was stirred at RT overnight. More (2,4-dimethoxybenzyl)amine (802.9 mg, 723 μL, 4.8 mmol, 1.1 equiv.) was added and the reaction mixture was again stirred at RT overnight. Then, it was partitioned between water and EtOAc. The organic layer was dried over MgSO₄; then filtered and concentrated in vacuo. The crude product was purified by silica gel chromatography using a heptane/ethyl acetate gradient as eluent to provide the title compound (1.85 g, quant.) as yellow oil. LC-MS: m/z=392.2 [M+H]⁺, ESI pos.

### Step 3: 1-(3-Acetyl-6-amino-2-pyridyl)-5-methyl-pyrazole-3-carbonitrile

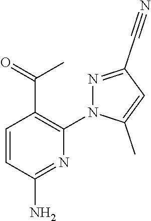 [SMILES: CC(=O)c1ccc(N)nc1-n1nc(C#N)cc1C]

Under an argon atmosphere, 1-[3-acetyl-6-[(2,4-dimethoxybenzyl)amino]-2-pyridyl]-5-methyl-pyrazole-3-carbonitrile (1.95 g, 4.73 mmol, 1 equiv.) was dissolved in DCM, extra dry (14.25 mL). TFA (10.25 g, 6.93 mL, 89.92 mmol, 19 equiv.) was added, and the reaction mixture was stirred at RT over 30 minutes turning into a dark red solution. Then, it was neutralised with trietylamine and concentrated. The residue was tritured in MeOH overnight and then filtered. The filtrate was concentrated. The crude product was purified by reverse phase chromatography with NH₃ to give the title compound (830 mg, 72%) as light brown powder. LC-MS: m/z=242.1 [M+H]⁺, ESI pos.

### Step 4: 2-Bromo-5-fluoro-1-oxido-pyridin-1-ium

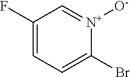

Under an argon atmosphere, 2-bromo-5-fluoro-pyridine (CAS 41404-58-4; 4.12 g, 22.9 mmol, 1 equiv.) was dissolved in DCM (41.2 mL). At 0° C., TFAA (14.5 g, 9.7 mL, 68.8 mmol, 3 equiv.) was added, followed by the dropwise addition over 30 minutes of H₂O₂ (2.68 g, 2.4 mL, 27.5 mmol, 1.2 equiv.): very exothermic! The reaction mixture was stirred at RT overnight, then carefully poured into ice cold water. The aqueous layer was extracted several times with DCM. The combined organic layers were dried over MgSO₄ and concentrated in vacuo at RT. The compound started to solidify under heat formation and was therefore cooled in an ice bath for 30 minutes. The crude was used at the next step without any further purification.

Dynamic DSC scan (30-500° C., 10° C./min): onset temperature for exothermic decomposition 110° C., enthalpy: 490 J/g.

### Step 5: 2-Bromo-5-fluoro-4-nitro-pyridine

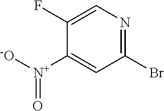

Under an argon atmosphere, 2-bromo-5-fluoro-1-oxido-pyridin-1-ium (4.4 g, 18.33 mmol, 1 equiv.) was dissolved in concentrated H₂SO₄ (28.2 g, 15.3 mL, 287 mmol, 15.6 equiv.) to give a pink solution. At 0° C., fuming HNO₃ (10.8 g, 7.65 mL, 171.2 mmol, 9.3 equiv.) was added slowly (exotherm: <10° C.), yielding a pale yellow solution. The reaction mixture was heated to 120° C. over 12 hours. The developing nitrogen oxides fumes were trapped in cooled aqueous NaOH solution.

After cooling to RT, the reaction mixture was poured onto ice cold water, brought to pH 6 with 4M NaOH and extracted with DCM. The combined organic extracts were dried over MgSO₄ and concentrated. The crude product was purified by silica gel chromatography using a heptane/ethyl acetate gradient as eluent and with a second silica gel chromatography using DCM/MeOH gradient as eluent to provide the title compound (1.33 g, 33%) as yellow oil.

Dynamic DSC scan (30-400° C., 10° C./min): Tm=148° C.: onset of temperature for exothermic decomposition: ca. 173° C., enthalpy: 1594 J/g. Highly energetic destruction!

### Step 6: 1-[3-Acetyl-6-[(6-bromo-4-nitro-3-pyridyl)amino]-2-pyridyl]-5-methyl-pyrazole-3-carbonitrile

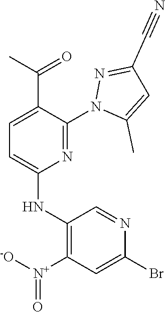

Under an argon atmosphere, 1-(3-acetyl-6-amino-2-pyridyl)-5-methyl-pyrazole-3-carbonitrile (650 mg, 2.7 mmol, 1 equiv.) was dissolved in DMF, extra dry (10.8 mL). At −10° C., NaH (168.1 mg, 3.5 mmol, 1.3 equiv.) was added (gas evolution) followed by a solution containing 2-bromo-5-fluoro-4-nitro-pyridine (654.9 mg, 2.96 mmol, 1.1 equiv.) in DMF, extra dry (5.4 mL). The reaction mixture turned black and was stirred at −10° C. over 30 minutes. A second portion of 2-bromo-5-fluoro-4-nitro-pyridine (178.6 mg, 0.81 mmol, 0.3 equiv.) was added, and the reaction mixture was stirred at −10° C. over 15 minutes. Then, it was partitioned between NH₄Cl solution and EtOAc. The organic layer was dried over MgSO₄, filtered; concentrated in vacuo. The crude product was purified by silica gel chromatography using a DCM/MeOH gradient as eluent to provide the title compound (520 mg, 43%) as orange gum. LC-MS: m/z=442.1 [M+H]⁺, ESI pos.

### Step 7: 1-[3-Acetyl-6-[(4-amino-6-bromo-3-pyridyl)amino]-2-pyridyl]-5-methyl-pyrazole-3-carbonitrile

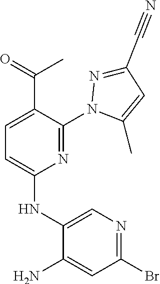 [SMILES: CC(=O)c1ccc(Nc2cnc(Br)cc2N)nc1-n1nc(C#N)cc1C]

Under an argon atmosphere and in a flask equipped with a mechanic stirrer, 1-[3-acetyl-6-[(6-bromo-4-nitro-3-pyridyl)amino]-2-pyridyl]-5-methyl-pyrazole-3-carbonitrile (0.92 g, 2.1 mmol, 1 equiv.) was suspended in ethanol (15.3 mL). Activated zinc (1.36 g, 20.8 mmol, 10 equiv.) was added, and the orange suspension was cooled to 0° C. A solution containing acetic acid (874 mg, 834 μL, 14.6 mmol, 7 equiv.) in ethanol (7.7 mL) was added dropwise while the temperature was kept under 5° C. The reaction mixture became black and was stirred at 0° C. over 30 minutes, then for 1 hour at RT. The reaction mixture was filtered, and the cake was washed with EtOAc. The filtrate was concentrated to obtain the crude product which was used in the next step without further purification. LC-MS: m/z=413.1 [M+H]+, ESI pos

### Step 8: 1-[3-Acetyl-6-(6-bromoimidazo[4,5-c]pyridin-3-yl)-2-pyridyl]-5-methyl-pyrazole-3-carbonitrile

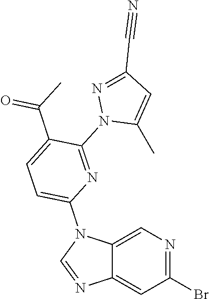 [SMILES: CC(=O)c1ccc(-n2cnc3cc(Br)ncc32)nc1-n1nc(C#N)cc1C]

Under an argon atmosphere, 1-[3-acetyl-6-[(4-amino-6-bromo-3-pyridyl)amino]-2-pyridyl]-5-methyl-pyrazole-3-carbonitrile (0.86 g, 2.1 mmol, 1 equiv.) was dissolved in trimethoxymethane (14.47 g, 14.9 mL, 136.4 mmol, 65.5 equiv.). The reaction mixture was stirred at 90° C. for 4 hours. The volatiles was evaporated. The crude product was purified by chromatography on silica gel using a DCM/MeOH gradient as eluent and a second chromatography on silica gel using a heptane/ethyl acetate gradient as eluent to provide the title compound (113 mg, 13%) as red solid. LC-MS: m/z=424.1 [M+H]⁺, ESI pos.

### Step 9: 1-[3-Acetyl-6-[6-[(6-methyl-3-pyridyl)amino]imidazo[4,5-c]pyridin-3-yl]-2-pyridyl]-5-methyl-pyrazole-3-carbonitrile

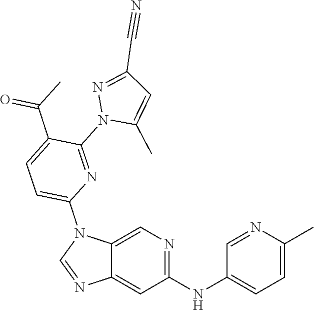 [SMILES: CC(=O)c1ccc(-n2cnc3cc(Nc4ccc(C)nc4)ncc32)nc1-n1nc(C#N)cc1C]

In analogy to the procedure described in example 1, step 2, using (6-methyl-3-pyridyl)amine as amine component, the title compound was obtained as yellow oil. LC-MS: m/z=450.3 [M+H]⁺, ESI pos.

### Step 10: 1-[3-(1-Hydroxyethyl)-6-[6-[(6-methyl-3-pyridyl)amino]imidazo[4,5-c]pyridin-3-yl]-2-pyridyl]-5-methyl-pyrazole-3-carbonitrile, Formic Acid

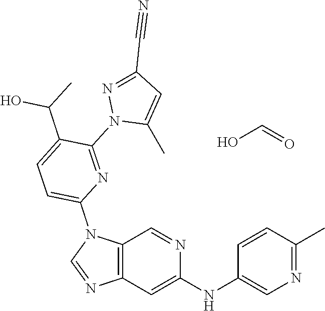 [SMILES: Cc1ccc(Nc2cc3ncn(-c4ccc(C(C)O)c(-n5nc(C#N)cc5C)n4)c3cn2)cn1 ; O=CO]

Under an argon atmosphere, 1-[3-acetyl-6-[6-[(6-methyl-3-pyridyl)amino]imidazo[4,5-c]pyridin-3-yl]-2-pyridyl]-5-methyl-pyrazole-3-carbonitrile (3 mg, 0.007 mmol, 1 equiv.) was dissolved in MeOH (0.165 mL). NaBH₄ (757 ug, 0.020 mmol, 3 equiv.) was added. The reaction mixture was stirred at RT over 30 minutes, then quenched by the addition of water. The reaction mixture was purified by reversed phase chromatography using 0.1% aqueous formic acid/acetonitrile as euluent to provide the title compound (1 mg, 29%) as white lyophilized powder. LC-MS: m/z=496.4 [M−H]⁻, ESI neg.

### Example 4

### 1-[6-[6-[(6-Methylpyridazin-3-yl)amino]imidazo[4,5-c]pyridin-3-yl]-2-[3-methyl-1-(2,2,2-trifluoroethyl)pyrazol-4-yl]-3-pyridyl]ethanol

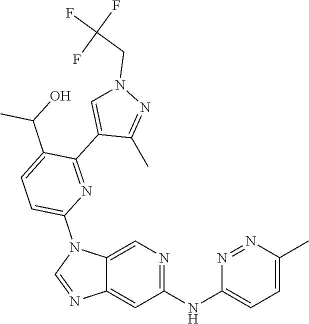 [SMILES: Cc1ccc(Nc2cc3ncn(-c4ccc(C(C)O)c(-c5cn(CC(F)(F)F)nc5C)n4)c3cn2)nn1]

### Step 1: 4-Bromo-3-methyl-1-(2,2,2-trifluoroethyl)pyrazole

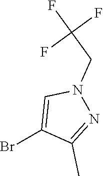

A mixture of 4-bromo-3-methylpyrazole (3:1 mixture with regioisomer; 15.0 g, 93.2 mmol, 1.0 equiv), 2,2,2-trifluoroethyl trifluoromethanesulfonate (22.71 g, 97.8 mmol, 1.05 equiv.) and CS₂CO₃ (25.33 g, 186.3 mmol, 2.0 equiv.) in DMF (150 mL) was stirred at 100° C. for 12 hours. Then, the reaction mixture was filtered. The filtrate was diluted with H₂O and extracted with ethyl acetate. The combined organic layers were washed with saturated aqueous NaCl solution, dried over anhydrous Na₂SO₄, filtered and concentrated. The crude product was purified by silica gel chromatography to give a 2:1-mixture of the title compound and its regioisomer 4-bromo-3-methyl-1-(2,2,2-trifluoroethyl)pyrazole (19.5 g, 80.2 mmol, 86%) as off-white oil LC-MS: m/z=242.9 [M+H]⁺, ESI pos.

¹H NMR (400 MHz, DMSO-d₆) δ=8.00 (s, 1H), 5.09-4.99 (m, 2H), 2.14 (s, 3H).

### Step 2: 3-Methyl-4-(4,4,5,5-tetramethyl-1,3,2-dioxaborolan-2-yl)-1-(2,2,2-trifluoroethyl)pyrazole

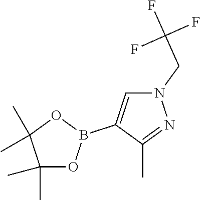 [SMILES: Cc1nn(CC(F)(F)F)cc1B1OC(C)(C)C(C)(C)O1]

To a mixture of bis(pinacolato)diboron (17.24 g, 67.9 mmol, 1.5 equiv.) and a 2:1 mixture of 4-bromo-3-methyl-1-(2,2,2-trifluoroethyl)pyrazole and 4-bromo-5-methyl-1-(2,2,2-trifluoroethyl)pyrazole (11.0 g, 45.26 mmol, 1.0 equiv.) in 1,4-dioxane (200 mL) was added Pd(dppf)Cl₂·CH₂Cl₂ (3.7 g, 4.53 mmol, 0.1 equiv.) and potassium acetate (5.66 mL, 90.53 mmol, 2.0 equiv.). The reaction mixture was stirred at 100° C. for 16 hours under an nitrogen atmosphere, then concentrated. The crude product was purified by flash chromatography on silica gel using a petroleum ether/ethyl acetate gradient as eluent (0%-20%) and by a second chromatography on by preparative HPLC using a hexanes/ethyl acetate gradient as eluent to give a first crop of the title compound (3:1 mixture of isomers; 6.0 g, 20.7 mmol, 33% yield) as off-white oil and a second crop of the title compound (2:1 mixture of isomers (2.0 g, 6.9 mmol, 11%) as yellow oil. LC-MS: m/z=291.1 [M+H]⁺, ESI pos.

### Step 3: 1-[6-Chloro-2-[3-methyl-1-(2,2,2-trifluoroethyl)pyrazol-4-yl]-3-pyridyl]ethanone

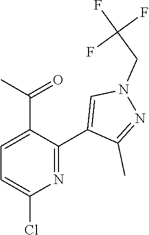 [SMILES: CC(=O)c1ccc(Cl)nc1-c1cn(CC(F)(F)F)nc1C]

A mixture of 3-methyl-4-(4,4,5,5-tetramethyl-1,3,2-dioxaborolan-2-yl)-1-(2,2,2-trifluoroethyl)-pyrazole (1.0 g, 3.45 mmol, 1.0 equiv.), 1-(2-bromo-6-chloro-3-pyridyl)ethanone (808 mg, 3.45 mmol, 1.0 eq), potassium carbonate (953 mg, 6.89 mmol, 2.0 eq) and Pd(dppf)Cl₂·CH₂Cl₂ (281 mg, 0.34 mmol, 0.1 equiv.) in 1,4-dioxane (20 mL) and water (2 mL) was stirred at 80° C. under a nitrogen atmosphere for 12 hours. The reaction mixture was concentrated. The crude product was purified by column on silica gel using as petroleum ether/ethyl acetate gradient as eluent to provide the title compound (790.0 mg, 2.29 mmol, 72% yield) as yellow oil. LC-MS: m/z=318.1, [M+H]⁺, ESI pos.

### Step 4a: N-(6-Methylpyridazin-3-yl)-3-(2-trimethylsilylethoxymethyl)imidazo[4,5-c]pyridin-6-amine

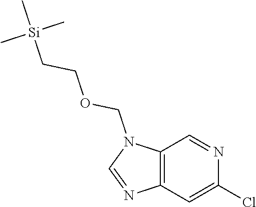 [SMILES: C[Si](C)(C)CCOCn1cnc2cc(Cl)ncc21]

To a solution of 6-chloro-3H-imidazo[4,5-c]pyridine (880.0 mg, 5.73 mmol, 1.0 equiv.) in DMF (10 mL) was added 2-(trimethylsilyl)ethoxymethyl chloride (2.03 mL, 11.5 mmol, 2.0 equiv.) and TEA (1.59 mL, 11.46 mmol, 2.0 equiv.). The mixture was stirred at 20° C. for 2 hours. Then, water was added, followed by extraction with ethyl acetate. The combined organics were washed with NaCl solution, then concentrated under reduced pressure to give a residue. The crude product was purified by column on silica using a petroleum ether/ethyl acetate gradient as eluent to provide the title compound (1.1 g, 54%) as yellow oil. LC-MS: m/z=284.0, [M+H]⁺, ESI pos.

### Step 5: N-(6-Methylpyridazin-3-yl)-3-(2-trimethylsilylethoxymethyl)imidazo[4,5-c]pyridin-6-amine

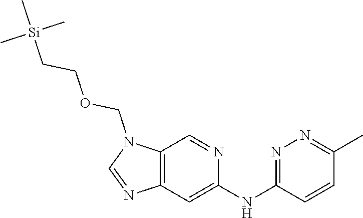

A mixture of 2-[(6-chloroimidazo[4,5-c]pyridin-3-yl)methoxy]ethyl-trimethyl-silane (900.0 mg, 3.17 mmol, 1.0 equiv.), 3-amino-6-methylpyridazine (1.04 g, 9.51 mmol, 3.0 eq), Cs₂CO₃ (2.06 g, 6.3 mmol, 2.0 equiv.) and allyl(trifluoromethylsulfonyloxy)palladiumditert-butyl-[3,6-dimethoxy-2-(2,4,6-triisopropylphenyl)phenyl]phosphane (248 mg, 0.32 mmol, 0.1 equiv.) in 1,4-dioxane (30 mL) was stirred at 100° C. under a nitrogen atmosphere for 12 hours. The mixture was filtered and concentrated. The crude product was purified by reversed phase flash using a acetonitrile/0.1% aqueous formic acid gradient as eluent to give the title compound (670.0 mg, 1.88 mmol, 59% yield) as a yellow solid. LC-MS: m/z=357.0, [M+H]⁺, ESI pos.

### Step 5: N-(6-Methylpyridazin-3-yl)-3H-imidazo[4,5-c]pyridin-6-amine

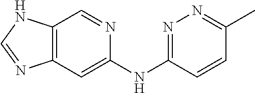 [SMILES: Cc1ccc(Nc2cc3nc[nH]c3cn2)nn1]

A solution of N-(6-methylpyridazin-3-yl)-3-(2-trimethylsilylethoxymethyl)imidazo[4,5-c]pyridin-6-amine (670 mg, 1.88 mmol, 1.0 equiv.) in TFA (7.0 mL) was stirred at 25° C. for 2 hours. The mixture was concentrated. The crude product was purified by reversed phase flash using a acetonitrile/0.1% aqueous formic acid gradient as eluent to give the title compound (580 mg, 2.56 mmol, 95% yield) as a yellow solid. LC-MS: m/z=227.2, [M+H]⁺, ESI pos.

¹H NMR (400 MHz, DMSO-d₆) δ=11.59-11.31 (m, 1H), 8.89 (s, 1H), 8.55 (s, 1H), 7.84-7.74 (m, 3H), 2.57 (s, 3H).

### Step 6: 1-[6-[6-[(6-Methylpyridazin-3-yl)amino]imidazo[4,5-c]pyridin-3-yl]-2-[3-methyl-1-(2,2,2-trifluoroethyl)pyrazol-4-yl]-3-pyridyl]ethanone

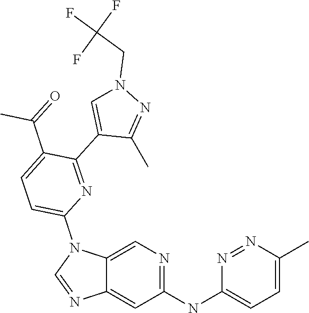 [SMILES: CC(=O)c1ccc(-n2cnc3cc(Nc4ccc(C)nn4)ncc32)nc1-c1cn(CC(F)(F)F)nc1C]

To a solution of N-(6-methylpyridazin-3-yl)-3H-imidazo[4,5-c]pyridin-6-amine (200.0 mg, 0.62 mmol, 1.0 equiv.) in DMSO (5 mL) was added 1-[6-chloro-2-[3-methyl-1-(2,2,2-trifluoroethyl)pyrazol-4-yl]-3-pyridyl]ethanone (197 mg, 0.62 mmol, 1.0 eq) and potassium carbonate (171 mg, 1.24 mmol, 2.0 eq).

The mixture was stirred at 70° C. for 12 hours, then quenched with water and filtrated. The filtrate was extracted with ethyl acetate. The organic layer was washed with brine, dried over with anhydrous sodium sulfate and concentrated. The residue was purified by reversed phase flash using a acetonitrile/0.1% aqueous formic acid gradient to provide a mixture of 1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanone, 1-[6-[6-[(6-methyl-pyridazin-3-yl)amino]imidazo[4,5-c]pyridin-3-yl]-2-[5-methyl-1-(2,2,2-trifluoroethyl)pyrazol-4-yl]-3-pyridyl]ethanone, 1-[6-[6-[(6-methyl-pyridazin-3-yl)amino]imidazo[4,5-c]pyridin-1-yl]-2-[3-methyl-1-(2,2,2-trifluoroethyl)pyrazol-4-yl]-3-pyridyl]ethanone and 1-[6-[6-[(6-methyl-pyridazin-3-yl)amino]imidazo[4,5-c]pyridin-1-yl]-2-[5-methyl-1-(2,2,2-trifluoroethyl)pyrazol-4-yl]-3-pyridyl]ethanone (150.0 mg, 0.3 mmol, 48% yield) as yellow solid. LC-MS: m/z=508.1, [M+H]⁺, ESI pos.

The mixture of the 4 isomers was purified by SFC (0.1% NH₃H₂O in isopropyl alcohol) to give two mixtures of two isomers each.

1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanone and 1-[6-[6-[(6-methylpyridazin-3-yl)amino]imidazo[4,5-c]pyridin-3-yl]-2-[5-methyl-1-(2,2,2-trifluoroethyl)pyrazol-4-yl]-3-pyridyl]ethanone (70 mg).

1-[6-[6-[(6-methylpyridazin-3-yl)amino]imidazo[4,5-c]pyridin-1-yl]-2-[3-methyl-1-(2,2,2-trifluoroethyl)pyrazol-4-yl]-3-pyridyl]ethanone and 1-[6-[6-[(6-methylpyridazin-3-yl)amino]imidazo[4,5-c]pyridin-1-yl]-2-[5-methyl-1-(2,2,2-trifluoroethyl)pyrazol-4-yl]-3-pyridyl]ethanone (50 mg).

LC-MS: m/z=508.3[M+H]⁺, ESI pos.

The mixture of 1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanone and 1-[6-[6-[(6-methylpyridazin-3-yl)amino]imidazo[4,5-c]pyridin-3-yl]-2-[5-methyl-1-(2,2,2-trifluoroethyl)pyrazol-4-yl]-3-pyridyl]ethanone (70.0 mg) was separated by SFC (0.1% NH₃H₂O in isopropyl alcohol) to give the title compound 1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanone (25 mg) as a yellow solid. LC-MS: m/z=508.2, [M+H]⁺, ESI pos.

¹H NMR (400 MHz, DMSO-d₆) δ=9.94 (s, 1H), 9.32 (d, J=0.8 Hz, 1H), 9.29 (s, 1H), 8.36 (d, J=0.6 Hz, 1H), 8.29 (d, J=8.6 Hz, 1H), 8.09 (s, 1H), 8.07 (d, J=8.6 Hz, 1H), 7.72 (d, J=9.1 Hz, 1H), 7.40 (d, J=9.1 Hz, 1H), 5.21-5.10 (m, 2H), 2.51 (br s, 3H), 2.36 (s, 3H), 2.30 (s, 3H)

and

1-[6-[6-[(6-methylpyridazin-3-yl)amino]imidazo[4,5-c]pyridin-3-yl]-2-[5-methyl-1-(2,2,2-trifluoroethyl)pyrazol-4-yl]-3-pyridyl]ethanone (12 mg) as a white solid. LC-MS: m/z=508.2, [M+H]⁺, ESI pos.

¹H NMR (400 MHz, DMSO-d₆) δ=10.04 (s, 1H), 9.01 (s, 1H), 8.81 (s, 1H), 8.77 (s, 1H), 8.35 (d, J=8.5 Hz, 1H), 7.95 (d, J=8.5 Hz, 1H), 7.90 (s, 1H), 7.74 (d, J=9.1 Hz, 1H), 7.41 (d, J=9.1 Hz, 1H), 5.26-5.14 (m, 2H), 2.52 (br s, 3H), 2.41 (s, 3H), 2.41 (s, 3H)

The mixture of 1-[6-[6-[(6-methylpyridazin-3-yl)amino]imidazo[4,5-c]pyridin-1-yl]-2-[3-methyl-1-(2,2,2-trifluoroethyl)pyrazol-4-yl]-3-pyridyl]ethanone and 1-[6-[6-[(6-methylpyridazin-3-yl)amino]imidazo[4,5-c]pyridin-1-yl]-2-[5-methyl-1-(2,2,2-trifluoroethyl)pyrazol-4-yl]-3-pyridyl]ethanone (50 mg) was purified by SFC (0.1% NH₃H₂O in isopropyl alcohol) to give

1-[6-[6-[(6-methylpyridazin-3-yl)amino]imidazo[4,5-c]pyridin-1-yl]-2-[3-methyl-1-(2,2,2-trifluoroethyl)pyrazol-4-yl]-3-pyridyl]ethanone and (35 mg) as a white solid. LC-MS: m/z=508.2, [M+H]⁺, ESI pos.

¹H NMR (400 MHz, DMSO-d₆) δ=10.03 (s, 1H), 9.08 (s, 1H), 9.02 (s, 1H), 8.78 (s, 1H), 8.54 (s, 1H), 8.37 (d, J=8.4 Hz, 1H), 7.97 (d, J=8.5 Hz, 1H), 7.63 (d, J=9.1 Hz, 1H), 7.45 (d, J=9.1 Hz, 1H), 5.24-5.13 (m, 2H), 2.54 (s, 3H), 2.44 (s, 3H), 2.14 (s, 3H)

and

1-[6-[6-[(6-methylpyridazin-3-yl)amino]imidazo[4,5-c]pyridin-1-yl]-2-[5-methyl-1-(2,2,2-trifluoroethyl)pyrazol-4-yl]-3-pyridyl]ethanone (18 mg) as a yellow solid. LC-MS: m/z=508.2, [M+H]⁺, ESI pos.

¹H NMR (400 MHz, DMSO-d₆) δ=9.94 (s, 1H), 9.33 (s, 1H), 9.31 (s, 1H), 8.38 (s, 1H), 8.34 (d, J=8.5 Hz, 1H), 8.07 (d, J=8.6 Hz, 1H), 7.75 (s, 1H), 7.70 (d, J=9.1 Hz, 1H), 7.40 (d, J=9.1 Hz, 1H), 5.28-5.18 (m, 2H), 2.53-2.51 (m, 3H), 2.41 (s, 3H), 2.40 (s, 3H).

### Step 7: 1-[6-[6-[(6-Methylpyridazin-3-yl)amino]imidazo[4,5-c]pyridin-3-yl]-2-[3-methyl-1-(2,2,2-trifluoroethyl)pyrazol-4-yl]-3-pyridyl]ethanol, Formic Acid

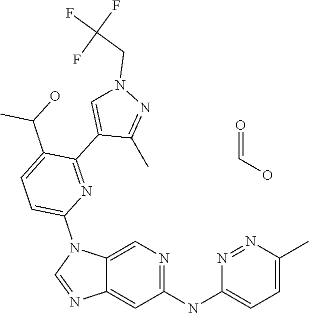 [SMILES: Cc1ccc(Nc2cc3ncn(-c4ccc(C(C)O)c(-c5cn(CC(F)(F)F)nc5C)n4)c3cn2)nn1 ; O=CO]

To a solution of 1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanone (23.0 mg, 0.05 mmol, 1.0 eq) in methanol (2 mL) was added NaBH₄ (8.57 mg, 0.23 mmol, 5.0 equiv.) at −70° C. The mixture was stirred at −70° C. for 0.5 hour. The reaction mixture was quenched by addition of aqueous NH₄Cl at 20° C. The crude product was purified by reversed phase HPLC using 0.1% aqueous formic acid/acetonitrile as eluent to provide the title compound (11.0 mg, 0.02 mmol, 46% yield) as a yellow solid. LC-MS: m/z=510.3, [M+H]⁺, ESI pos.

¹H NMR (400 MHz, MeOH-d₄) δ=9.32 (s, 1H), 9.07 (s, 1H), 8.29 (d, J=8.5 Hz, 1H), 8.25 (s, 1H), 8.13 (s, 1H), 8.02 (s, 1H), 7.89 (d, J=8.5 Hz, 1H), 7.75 (d, J=9.3 Hz, 1H), 7.47 (d, J=9.1 Hz, 1H), 5.04-4.95 (m, 3H), 2.56 (s, 3H), 2.35 (s, 3H), 1.46 (d, J=6.4 Hz, 3H).

### Example 5

### 1-[3-acetyl-6-[6-[methyl-(6-methylpyridazin-3-yl)amino]imidazo[4,5-c]pyridin-3-yl]-2-pyridyl]-5-methyl-pyrazole-3-carbonitrile

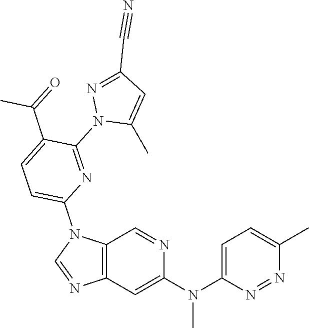 [SMILES: CC(=O)c1ccc(-n2cnc3cc(N(C)c4ccc(C)nn4)ncc32)nc1-n1nc(C#N)cc1C]

### Step 1: 1-[3-Acetyl-6-(6-chloroimidazo[4,5-c]pyridin-3-yl)-2-pyridyl]-5-methyl-pyrazole-3-carbonitrile (Corresponding to Example 1, Step 1)

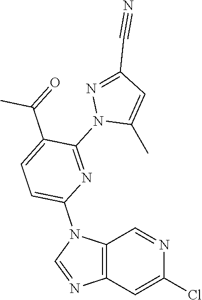 [SMILES: CC(=O)c1ccc(-n2cnc3cc(Cl)ncc32)nc1-n1nc(C#N)cc1C]

A solution of 6-chloro-3H-imidazo[4,5-c]pyridine (520 mg, 3.381 mmol, 1.0 equiv.), 1-(3-acetyl-6-chloro-2-pyridyl)-5-methyl-pyrazole-3-carbonitrile (971 mg, 3.7 mmol, 1.1 equiv.) in dimethyl sulfoxide (15.6 mL) with 1,8-diazabicyclo[5.4.0]undec-7-ene (567 mg, 561 μL, 3.72 mmol, 1.1 equiv.) was stirred at 80° C. for 3 hours. Again, 1,8-diazabicyclo[5.4.0]undec-7-ene (567 mg, 561 μL, 3.72 mmol, 1.1 equiv.) was added and stirring at 80° C. was continued for 6 hours. The reaction mixture was poured on water and extracted with EtOAc. The organic layers were dried over Na₂SO₄, filtered and concentrated. The crude product was purified by silica gel chromatography using a DCM/MeOH gradient as eluent to provide the title compound (456 mg, 36%) as light brown solid

### Step 2: 1-[3-acetyl-6-[6-[methyl-(6-methylpyridazin-3-yl)amino]imidazo[4,5-c]pyridin-3-yl]-2-pyridyl]-5-methyl-pyrazole-3-carbonitrile

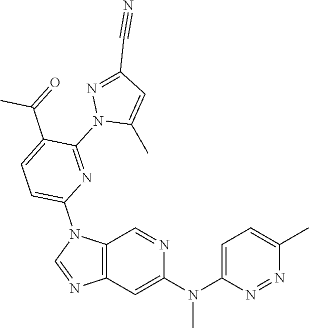 [SMILES: CC(=O)c1ccc(-n2cnc3cc(N(C)c4ccc(C)nn4)ncc32)nc1-n1nc(C#N)cc1C]

A suspension of 1-[3-acetyl-6-(6-chloroimidazo[4,5-c]pyridin-3-yl)-2-pyridyl]-5-methyl-pyrazole-3-carbonitrile (125 mg, 0.288 mmol, 1.0 equiv), methyl-(6-methylpyridazin-3-yl)amine (CAS 18591-82-7; 73.4 mg, 0.595 mmol, 2.0 equiv.) and Cs₂CO₃ (485.1 mg, 1.488 mmol, 5.0 equiv.) at RT in 1,4-dioxane (8.2 mL) was flushed with argon for 5 minutes. Then [tBuBrettPhos Pd(allyl)]OTf (23.3 mg, 0.03 mmol, 0.10 equiv.) was added. The vial was sealed. The reaction mixture was heated to 80° C. for 2 hours. Again [tBuBrettPhos Pd(allyl)]OTf (23.26 mg, 0.03 mmol, 0.10 equiv.) was added and stirring at 80° C. was continued for 2 hours. Then, the reaction mixture was concentrated. The crude product was purified by silica gel chromatography using a DCM/MeOH gradient and a second silica gel chromatography using a EtOAc/MeOH gradient as eluent to provide the title compound (33 mg, 30%) as white solid. LC-MS: m/z=242.1 [M+H]⁺, ESI pos.

### Example 6

### 1-[3-Acetyl-6-[6-(3-methyl-6,7-dihydropyridazino[4,3-b][1,4]oxazin-8-yl)imidazo[4,5-c]pyridin-3-yl]-2-pyridyl]-5-methyl-pyrazole-3-carbonitrile

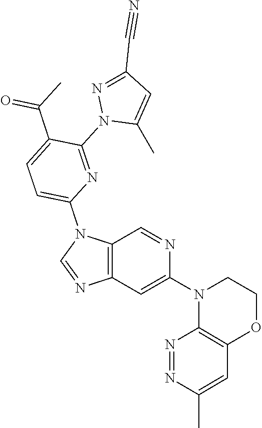 [SMILES: CC(=O)c1ccc(-n2cnc3cc(N4CCOc5cc(C)nnc54)ncc32)nc1-n1nc(C#N)cc1C]

### Step 1: 3,6-Dichloro-4-(2-iodoethoxy)pyridazine

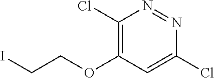 [SMILES: Clc1cc(OCCI)c(Cl)nn1]

To the solution of 3,4,6-trichloropyridazine (4.5 g, 24.5 mmol, 1 equiv.) in THF (100 mL) was added 2-iodoethanol (4.43 g, 25.8 mmol, 1.05 equiv.) at 0° C. under an nitrogen atmosphere. The reaction mixture was stirred at 0° C. for 30 minutes. Then, NaH (1.18 g, 29.44 mmol, 1.2 eq) was added at 0° C. The reaction mixture was stirred at 50° C. for 16 hours, then taken up in saturated aqueous NH₄Cl solution and extracted with EtOAc. The combined organic layer was dried over Na₂SO₄, filtered and concentrated. The crude product was purified by silica gel chromatography using a petroleum ether/ethyl acetate gradient as eluent to provide the title compound (3 g, 9.41 mmol, 38% yield) as light yellow solid. LC-MS: m/z=318.9 [M+H]⁺, ESI pos.

### Step 2: 2-(3,6-Dichloropyridazin-4-yl)oxy-N-[(2,4-dimethoxyphenyl)methyl]ethanamine

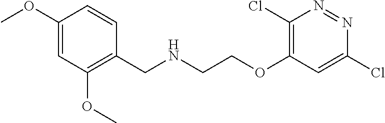 [SMILES: COc1ccc(CNCCOc2cc(Cl)nnc2Cl)c(OC)c1]

To a solution of 3,6-dichloro-4-(2-iodoethoxy)pyridazine (3.0 g, 9.41 mmol, 1 equiv.) and 2,4-dimethoxybenzylamine (1.7 mL, 11.3 mmol, 1.2 equiv.) in DMSO (30 mL) was added DIPEA (3.65 g, 28.2 mmol, 3 equiv.). The mixture was stirred at 50° C. for 12 hours, then poured into water and extracted with EtOAc. The combined organic layers were washed with brine, dried over Na₂SO₄, filtered and concentrated. The crude product was purified by silica gel chromatography using a petroleum ether/ethyl acetate gradient as eluent to provide the title compound (1.25 g, 3.5 mmol, 37% yield) as yellow solid. LC-MS: m/z=358.2 [M+H]⁺, ESI pos.

### Step 3: 3-Chloro-8-[(2,4-dimethoxyphenyl)methyl]-6,7-dihydropyridazino[4,3-b][1,4]oxazine

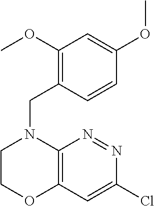 [SMILES: COc1ccc(CN2CCOc3cc(Cl)nnc32)c(OC)c1]

To a solution of Cs₂CO₃ (2.55 g, 7.82 mmol, 2 equiv.) and 2-(3,6-dichloropyridazin-4-yl)oxy-N-[(2,4-dimethoxyphenyl)methyl]ethanamine (1.4 g, 3.91 mmol, 1 equiv.) in toluene (40 mL) was added BINAP (340.7 mg, 0.55 mmol, 0.14 equiv.). The mixture was stirred at 110° C. for 2 hours, then diluted with DCM and filtered through a pad of celite. The filtrate was washed with brine. The organic layers were dried over Na₂SO₄, filtered and concentrated. The crude product was purified by silica column chromatography using a petroleum ether/ethyl acetate gradient (900 mg, 2.8 mmol, 72% yield) as yellow solid. LC-MS: m/z=322.1 [M+H]⁺, ESI pos.

¹H NMR (400 MHz, CDCl₃-d) δ=7.03 (d, J=8.3 Hz, 1H), 6.65 (s, 1H), 6.53-6.43 (m, 2H), 4.40 (s, 2H), 4.39-4.35 (m, 2H), 3.82 (s, 6H), 3.49-3.44 (m, 2H).

### Step 4: 8-[(2,4-Dimethoxyphenyl)methyl]-3-methyl-6,7-dihydropyridazino[4,3-b][1,4]oxazine

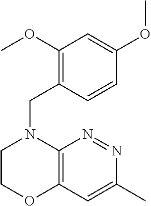 [SMILES: COc1ccc(CN2CCOc3cc(C)nnc32)c(OC)c1]

To a solution of trimethylboroxine (971.4 mg, 3.87 mmol, 1.5 equiv.) and 3-chloro-8-[(2,4-dimethoxyphenyl)methyl]-6,7-dihydropyridazino[4,3-b][1,4]oxazine (830.0 mg, 2.58 mmol, 1 equiv.) in 1,4-dioxane (15 mL) were added Pd(dppf)Cl₂ (189 mg, 0.26 mmol, 0.1 equiv.) and K₂CO₃ (713 mg, 5.16 mmol, 2 equiv.) under a nitrogen atmosphere. The mixture was stirred at 100° C. for 12 hours, then diluted with DMF (10 mL) and MeOH (40 mL). Thiourea on resin was added, and stirring was continued for 3 hours. The mixture was filtered and concentrated. The crude product was purified by prep-HPLC using an 0.1% aqueous formic acid/acetonitrile gradient as eluent. The product containing fraction were lyophilized to give the title compound (600 mg, 1.99 mmol, 77% yield) as yellow gum. LC-MS: m/z=302.1 [M+H]⁺, ESI pos.

### Step 5: 3-Methyl-7,8-dihydro-6H-pyridazino[4,3-b][1,4]oxazine

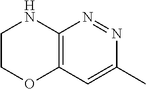

A solution of 3-chloro-8-[(2,4-dimethoxyphenyl)methyl]-6,7-dihydropyridazino[4,3-b][1,4]oxazine (550.0 mg, 1.71 mmol, 1 equiv.) in TFA (5.0 mL, 67.3 mmol, 39.5 equiv.) was stirred at 25° C. for 4 hours. The mixture was diluted with DCM, then basified by the addition of aqueous ammonia to bring the pH to 8-9. Then, the mixture was extracted with water. The aqueous layer was concentrated in vacuo. The residue was purified by prep-HPLC using a 10 mM aqueous NH₄CO₃ solution/acetonitrile gradient as eluent. The product-containing fractions were lyophilized to provide the title compound (88.8 mg, 0.59 mmol, 34% yield) as white solid. LC-MS: m/z=152.0 [M+H]⁺, ESI pos.

¹H NMR (400 MHz, MeOH-d₄) δ=6.54 (s, 1H), 4.41-4.34 (m, 2H), 3.50-3.42 (m, 2H), 2.36 (s, 3H).

### Step 6: 1-[3-Acetyl-6-[6-(3-methyl-6,7-dihydropyridazino[4,3-b][1,4]oxazin-8-yl)imidazo[4,5-c]pyridin-3-yl]-2-pyridyl]-5-methyl-pyrazole-3-carbonitrile

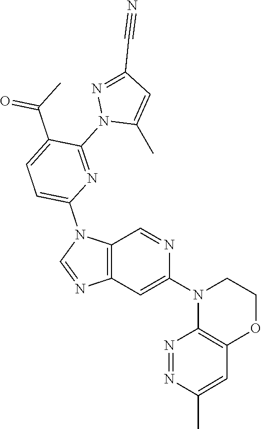 [SMILES: CC(=O)c1ccc(-n2cnc3cc(N4CCOc5cc(C)nnc54)ncc32)nc1-n1nc(C#N)cc1C]

A suspension of 1-[3-acetyl-6-(6-chloroimidazo[4,5-c]pyridin-3-yl)-2-pyridyl]-5-methyl-pyrazole-3-carbonitrile (example 1, step 1; 100 mg, 0.24 mmol, 1.0 equiv.), 3-methyl-7,8-dihydro-6H-pyridazino[4,3-b][1,4]oxazine (36.0 mg, 0.24 mmol, 1.0 equiv.) and cesium carbonate (388.1 mg, 1.19 mmol, 5.0 equiv.) at RT in 1,4-dioxane (6.56 mL) was flushed with argon for 5 minutes. Then [tBuBrettPhos Pd(allyl)]OTf (18.6 mg, 0.024 mmol, 0.10 equiv.) was added. The reaction vial was sealed, and the mixture was heated to 80° C. for 2 hours. Again, [tBuBrettPhos Pd(allyl)]OTf (18.61 mg, 0.024 mmol, 0.10 eq) were added and heating to 80° C. was continued for 2 hours. The reaction mixture was cooled to RT, again flushed with argon and treated with [tBuBrettPhos Pd(allyl)]OTf (18.61 mg, 0.024 mmol, 0.10 eq) and stirred for 1 hour at 80° C. The reaction mixture was concentrated. The crude product was purified by silica gel chromatography using a dichloromethane/MeOH gradient as eluent. The product-containing fractions were concentrated. The residue was triturated with diethylether. The solid was filtered and dried to provide the title compound (23 mg, 18%) as light brown solid. LC-MS: m/z=493.2 [M+H]⁺, ESI pos.

### Example 7

### 1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanol

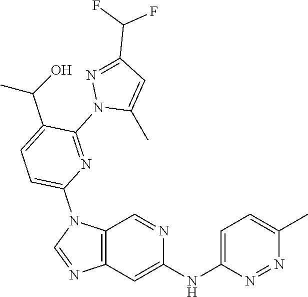 [SMILES: Cc1ccc(Nc2cc3ncn(-c4ccc(C(C)O)c(-n5nc(C(F)F)cc5C)n4)c3cn2)nn1]

### Step 1: 1-[6-Chloro-2-[3-(difluoromethyl)-5-methyl-pyrazol-1-yl]-3-pyridyl]ethanone

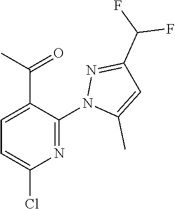 [SMILES: CC(=O)c1ccc(Cl)nc1-n1nc(C(F)F)cc1C]

A mixture of 1-(6-chloro-2-fluoro-3-pyridyl)ethanone (1.0 g, 5.76 mmol, 1.0 equiv.), 3-(difluoromethyl)-5-methyl-1H-pyrazole (761.13 mg, 5.76 mmol, 1.0 equiv.) and DIPEA (2.22 g, 17.3 mmol, 3.0 equiv.) in DMSO (10 mL) was stirred at 100° C. for 12 hours. The crude product was purified by reversed phase chromatography using an acetonitrile/0.1% aqueous formic acid gradient as eluent and lyophilized to afford the title compound (800 mg, 2.8 mmol, 49% yield) as a grey solid. LC-MS: m/z=286.1, [M+H]⁺, ESI pos.

### Step 2: 1-[2-[3-(Difluoromethyl)-5-methyl-pyrazol-1-yl]-6-[6-[(6-methylpyridazin-3-yl)amino]imidazo[4,5-c]pyridin-3-yl]-3-pyridyl]ethanone

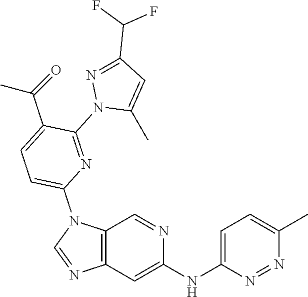 [SMILES: CC(=O)c1ccc(-n2cnc3cc(Nc4ccc(C)nn4)ncc32)nc1-n1nc(C(F)F)cc1C]

To a solution of N-(6-methylpyridazin-3-yl)-3H-imidazo[4,5-c]pyridin-6-amine (example 4, step 6; 230.0 mg, 0.71 mmol, 1.0 equiv.) in DMSO (5 mL) was added 1-[6-chloro-2-[3-(difluoromethyl)-5-methyl-pyrazol-1-yl]-3-pyridyl]ethanone (example 1, step 1; 203.3 mg, 0.71 mmol, 1.0 equiv.) and K₂CO₃ (196.7 mg, 1.42 mmol, 2.0 equiv.). The mixture was stirred at 70° C. for 12 hours, then diluted with water and extracted with EtOAc. The combined organic layers were washed with brine, dried over with anhydrous sodium sulfate and concentrated. The residue was purified twice by silica gel chromatography using a hexane/ethyl acetate gradient as eluent to give a mixture of regioisomers 1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanone and 1-[2-[3-(difluoromethyl)-5-methyl-pyrazol-1-yl]-6-[6-[(6-methylpyridazin-3-yl)amino]imidazo[4,5-c]pyridin-1-yl]-3-pyridyl]ethanone (30.0 mg, 0.06 mmol, 9% yield) as a yellow solid. LC-MS: m/z=476.2, [M+H]⁺, ESI pos.

This mixture was separated by SFC using a acetonitrile/MeOH+0.1% NH3 as and eluent to obtain the title compound 1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanone (10 mg, 32% yield) as a yellow solid. LC/MS: m/z=476.1, [M+H]⁺, ESI pos.

¹H NMR (400 MHz, DMSO-d₆) δ=9.99 (s, 1H), 9.33 (s, 1H), 9.22 (d, J=0.7 Hz, 1H), 8.44 (d, J=8.4 Hz, 1H), 8.34 (s, 1H), 8.29 (d, J=8.4 Hz, 1H), 7.76 (d, J=9.3 Hz, 1H), 7.41 (d, J=9.2 Hz, 1H), 7.22-6.90 (m, 1H), 6.76 (s, 1H), 2.59 (s, 3H), 2.54 (br s, 3H), 2.03 (s, 3H).

In addition, the regioisomer 1-[2-[3-(difluoromethyl)-5-methyl-pyrazol-1-yl]-6-[6-[(6-methylpyridazin-3-yl)amino]imidazo[4,5-c]pyridin-1-yl]-3-pyridyl]ethanone (2 mg, 0.02 mmol, 7% yield) as a yellow solid.

LC-MS: m/z=476.1, [M+H]⁺, ESI pos.

### Step 3: 1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanol

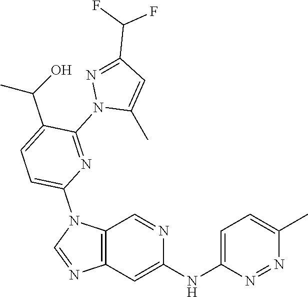 [SMILES: Cc1ccc(Nc2cc3ncn(-c4ccc(C(C)O)c(-n5nc(C(F)F)cc5C)n4)c3cn2)nn1]

To a solution of 1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanone (8.0 mg, 0.02 mmol, 1.0 equiv.) in MeOH (2 mL) was added a solution of NaBH₄ (3.18 mg, 0.08 mmol, 5.0 equiv.) in MeOH (0.5 mL) at −70° C. The mixture was stirred at −70° C. for 0.5 hour. The reaction mixture was quenched by addition of aqueous ammonium chloride solution at 20° C.

The crude product was purified reversed phase chromatography using an acetonitrile/0.1% aqueous formic acid gradient as eluent to give the title compound (1.2 mg, 14% yield) as a lyophilized white solid. LC-MS: m/z=478.2, [M+H]⁺, ESI pos.

¹H NMR (400 MHz, MeOH-d₄) δ=9.43 (s, 1H), 9.27 (s, 1H), 8.52 (d, J=8.5 Hz, 1H), 8.20 (d, J=8.5 Hz, 1H), 7.85 (s, 1H), 7.77 (s, 2H), 6.98-6.65 (m, 1H), 6.61 (s, 1H), 4.80-4.80 (m, 1H), 2.61 (s, 3H), 2.42 (s, 3H), 1.39 (d, J=6.4 Hz, 3H).

### Example 8—Phosphorylation Assay SIK1-3

In the presence of SIK2 (resp. SIK1 or SIK3) and ATP the CHK-peptide (KKKVSRSGLYRSPSMPENLNRPR with C-terminal arginine amide modification) were phosphorylated at one of the four feasible serine's. Only one phosphorylation is observed under the assay conditions. 60 nl of each compound dilution series (12 point; dilution factor 3, generally 30 μM to 170 pM) in DMSO were transferred by acoustic dispensing to the assay plate and 30 minutes pre-incubated (ambient temperature) after the addition of 5 μl SIK1 (5 nM) resp. 5 μl SIK2 (0.5 nM) or 7 μl SIK3 (1.5 nM) in assay-buffer (12.5 mM HEPES (pH 7.0), 10 mM magnesium acetate, 0.005% BSA). 10 μM CHK-peptide solution and 5 μl of 100 μM ATP for SIK1 & SIK2 resp. 3 μl for SIK3 in assay-buffer were added and incubated ambient for 45 minutes. 40 μl of 0.125% formic acid in water were added to quench the reaction. RapidFire (RF) Mass Spectrometry was utilized for data generation as described below. The multiple charged species (3-5 charges) for the phosphorylated and non-phosphorylated form measured by MRM (Multiple Reaction Monitoring; API5000 or 6500+) or EIC (Extracted Ion Current; QToF) were summed up and the ratio calculated (sum phosphorylated species/sum all species) for data evaluation. Normalization was performed by Genedata software based on the non-inhibition control DMSO and the commercially available STK inhibitor @1 μM YKL-05-099 (CAS number 1936529-65-5). The results of the assay are expressed in half-maximal inhibitory concentrations (IC50s) and are summarized below in Table 1.

### RapidFire Setup:

Samples were aspirated by vacuum for max. 600 ms and loaded to C4-cartridge (Agilent; #G9203A) for 3000 ms@1.5 ml/min with 0.1% formic acid in water. Afterwards samples were transferred to the API5000 (API6500+) or QToF mass spectrometer for 4000 ms@1.25 ml/min with 90% acetonitrile; 10% water; 0.007% TFA; 0.093 formic acid. The cartridge was reconditioned for additional 500 ms with 0.1% formic acid in water.

### MS-Setup Sciex AP15000/AP16500+:

All MS analyses using the following MS-setup in MRM mode: Electrospray positive; Ion Spray Voltage: 4000V; Temperature: 550° C.; Collision Gas: 5; Curtain Gas: 15; Gas 1: 40; Gas 2: 42; EP: 10. DP=declustering potential; CE=collision energy; CXP=cell exit potential.

| | Q1 | Q3 | Time | DP | CE | CXP |
|---|---|---|---|---|---|---|
| Name | (m/z) | (m/z) | (ms) | (V) | (V) | (V) |
| CHK(4+) | 676.0 | 84.2 | 50 | 46 | 99 | 18 |
| CHK(5+) | 541.0 | 84.2 | 50 | 51 | 71 | 36 |
| pCHK(4+) | 696.0 | 84.2 | 50 | 66 | 105 | 18 |
| pCHK(5+) | 557.0 | 84.2 | 50 | 51 | 53 | 4 |

### MS-Setup Agilent QToF 6545

All MS analyses using the following MS-setup in Mode MS: Dual AJS Electrospray positive; VCap: 3000V; Drying & Sheath gas: 340° C.@81/min; Nebulizer: 60 psig; Nozzle Voltage: 2000V; Fragmentor: 130V; Skimmer: 35V; Oct1 RF Vpp: 700V; Ref masses on@5 spectra/s

| Name | EIC (m/z) | Width |
|---|---|---|
| CHK(3+) | 900.8345 | 50 ppm |
| CHK(4+) | 675.8789 | 50 ppm |
| CHK(5+) | 540.9048 | 50 ppm |
| pCHK(3+) | 927.4961 | 50 ppm |
| pCHK(4+) | 695.8747 | 50 ppm |

TABLE 1
IC50 values for inhibition of SIK1, SIK2 and SIK3:

| | SIK1 RF IC50 | | |
|---|---|---|---|
| Example | (uM) | SIK2 RF IC50 (uM) | SIK3 RF IC50 (uM) |
| 1 | 4.524 | 0.042 | 0.121 |
| 2 | 0.366 | 0.005 | 0.003 |
| 3 | 0.078 | 0.022 | 0.022 |
| 4 | 0.396 | 0.003 | 0.003 |
| 5 | 19.024 | 0.687 | 2.056 |
| 6 | 17.415 | 2.542 | 3.286 |
| 7 | 0.879 | 0.016 | 0.007 |

### Example A

Film coated tablets containing the following ingredients for the tablet kernel and film coat can be manufactured in a conventional manner:

| | |
|---|---|
| Ingredients | Per tablet |

| | | | | |
|---|---|---|---|---|
| Kernel: | | | | |
| Compound of formula (I) or a | 10.0 | mg | 200.0 | mg |
| pharmaceutically acceptable salt thereof | | | | |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule | |
|---|---|---|
| Compound of formula (I) or a | 25.0 | mg |
| pharmaceutically acceptable salt thereof | | |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| Compound of formula (I) or a | 3.0 | mg |
|---|---|---|
| pharmaceutically acceptable salt thereof | | |
| Polyethylene glycol 400 | 150.0 | mg |
| Acetic acid | q.s. ad pH 5.0 | |
| Water for injection solutions | ad 1.0 | ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I) 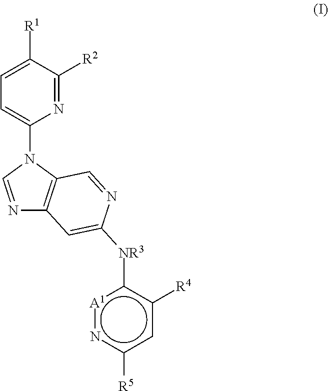
wherein
R¹ is hydroxyalkyl, alkylcarbonyl, cyclopropyl, haloalkyl or cyano;
R² is heteroaryl optionally substituted with one, two or three substituents independently selected from alkyl, haloalkyl, halogen, cyano, haloalkoxy and alkoxy;
R³ is hydrogen or alkyl;
R⁴ is hydrogen or alkyl; or R³ together with R⁴ and the atoms they are attached to, form a five- to seven-membered heterocycloalkyl or heteroaryl ring;
R⁵ is alkyl or haloalkyl; and
A¹ is —CH— or —N—;
or a pharmaceutically acceptable salt thereof.

2. A compound according to claim 1, wherein R¹ is hydroxyalkyl or alkylcarbonyl.

3. A compound according to claim 1, wherein R¹ is hydroxyethyl or methylcarbonyl.

4. A compound according to claim 1, wherein R² is cyano, haloalkyl or haloalkoxy.

5. A compound according to claim 1, wherein R² is cyano, difluoromethyl or difluoromethoxy.

6. A compound according to claim 1, wherein R³ is hydrogen or methyl, and R⁴ is hydrogen; or R³ together with R⁴ and the atoms they are attached to, form a five- to seven-membered heterocycloalkyl or heteroaryl ring.

7. A compound according to claim 1, wherein R³ is hydrogen or methyl, and R⁴ is hydrogen; or R³ together with R⁴ and the atoms they are attached to, form 3,4-dihydro-2H-1,4-oxazine.

8. A compound according to claim 1, wherein R⁵ is alkyl.

9. A compound according to claim 1, wherein R⁵ is methyl.

10. A compound according to claim 1, wherein A¹ is —N—.

11. A compound according to claim 1, wherein A¹ is —CH—.

12. A compound of formula (I) according to claim 1, selected from
1-[3-acetyl-6-[6-[(6-methylpyridazin-3-yl)amino]imidazo[4,5-c]pyridin-3-yl]-2-pyridyl]-5-methyl-pyrazole-3-carbonitrile;
1-[3-(1-hydroxyethyl)-6-[6-[(6-methylpyridazin-3-yl)amino]imidazo[4,5-c]pyridin-3-yl]-2-pyridyl]-5-methyl-pyrazole-3-carbonitrile;
1-[3-(1-hydroxyethyl)-6-[6-[(6-methyl-3-pyridyl)amino]imidazo[4,5-c]pyridin-3-yl]-2-pyridyl]-5-methyl-pyrazole-3-carbonitrile;
1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanol;
1-[3-acetyl-6-[6-[methyl-(6-methylpyridazin-3-yl)amino]imidazo[4,5-c]pyridin-3-yl]-2-pyridyl]-5-methyl-pyrazole-3-carbonitrile;
1-[3-acetyl-6-[6-(3-methyl-6,7-dihydropyridazino[4,3-b][1,4]oxazin-8-yl)imidazo[4,5-c]pyridin-3-yl]-2-pyridyl]-5-methyl-pyrazole-3-carbonitrile; and
1-[2-[3-(difluoromethyl)-5-methyl-pyrazol-1-yl]-6-[6-[(6-methylpyridazin-3-yl)amino]imidazo[4,5-c]pyridin-3-yl]-3-pyridyl]ethanol;
or a pharmaceutically acceptable salt thereof.

13. A compound of formula (I) according to claim 1, selected from
1-[3-acetyl-6-[6-[(6-methylpyridazin-3-yl)amino]imidazo[4,5-c]pyridin-3-yl]-2-pyridyl]-5-methyl-pyrazole-3-carbonitrile;
1-[3-(1-hydroxyethyl)-6-[6-[(6-methylpyridazin-3-yl)amino]imidazo[4,5-c]pyridin-3-yl]-2-pyridyl]-5-methyl-pyrazole-3-carbonitrile; and
1-[6-[6-[(6-methylpyridazin-3-yl)amino]imidazo[4,5-c]pyridin-3-yl]-2-[3-methyl-1-(2,2,2-trifluoroethyl)pyrazol-4-yl]-3-pyridyl]ethanol;
or a pharmaceutically acceptable salt thereof.

14. A process for the preparation of a compound according to a claim 1, comprising one of the following steps:
(a) the reaction of a compound of formula (B1) 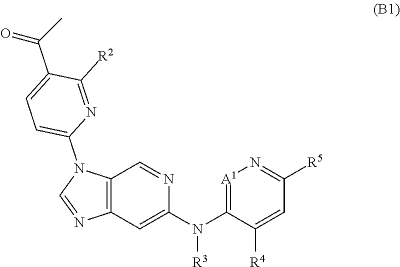
with a suitable reducing agent in presence of a suitable solvent,
(b) the reaction of a compound of formula (B2) 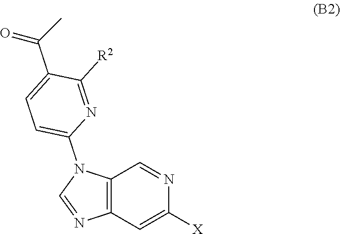
with a compound of formula (B3) 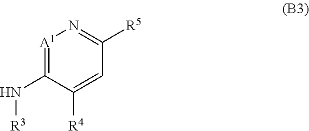
in presence of a suitable base, a suitable catalyst and a suitable solvent, or
(c) the reaction of a compound of formula (B4) 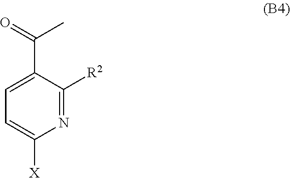
with a compound of formula (B5) 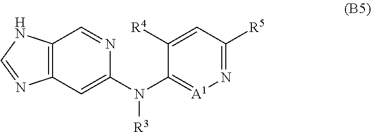
in presence of a suitable base,
wherein X is halogen; A¹, R², R³, R⁴ and R⁵ are as defined in any one of claims 1 to 9.

15. A pharmaceutical composition comprising a compound of formula (I) according to claim 1 or a pharmaceutically acceptable salt thereof, and a therapeutically inert carrier.

16. A method for the treatment or prophylaxis of rheumatoid arthritis, juvenile rheumatoid arthritis, non-alcoholic steatohepatitis (NASH), primary sclerosing cholangitis, giant cell vasculitis, inflammatory bowel diseases (IBD), atherosclerosis, type 2 diabetes or glomerulonephritis, which method comprises administering an effective amount of a compound of formula (I) according to claim 1 or a pharmaceutically acceptable salt thereof, to a patient in need thereof.
